(19) United States
(12) Patent Application Publication
(10) Pub. No.: US 2026/0091125 A1
(43) Pub. Date: Apr. 2, 2026

(54) MULTIFUNCTIONAL DELIVERY SYSTEM AND USES THEREOF

(71) Applicant: NANOCARRY THERAPEUTICS LTD., Ness Ziona (IL)

(72) Inventors: Revital MANDIL LEVIN, Ness Ziona (IL); Oshra BETZER, Elkana (IL); Yuval SAGIV, Gedera (IL); Adam ANTEBI, Modiin (IL); Rachela POPOVTZER, Givat Shmuel (IL)

(21) Appl. No.: 18/865,714

(22) PCT Filed: Jan. 12, 2023
(86) PCT No.: PCT/IL2023/050039

§ 371 Date: Nov. 14, 2024

Related U.S. Application Data
(60) Provisional application No. 63/342,174, filed on May 16, 2022.

(51) Int. Cl. A61K 47/69; A61K 47/62; A61K 47/68; A61P 35/00

(52) U.S. Cl. CPC A61K 47/6929; A61K 47/62; A61K 47/6851; A61P 35/00

(57) ABSTRACT

The invention provides multifunctional system for the synchronized delivery of distinct immunoglobulins, in particular antibodies and antibody fragments to specific cancerous cells and tissues located outside of the brain. The multifunctional system is based on an inorganic core particle which is conjugated through a first polymeric linker to a first immunoglobulin; through a second polymeric linker to a second immunoglobulin; through a third polymeric linker to a penetration enhancing moiety; and to a fourth, monofunctional capping linker or spacer. Further provided are a process for preparation of the multifunctional system, pharmaceutical compositions comprising the multifunctional system and uses thereof in therapeutic and/or diagnostic methods.

### FIELD OF INVENTION

The present invention is in the field of immunotherapy and relates to delivery systems for therapeutic and diagnostic uses.

### BACKGROUND OF THE INVENTION

Recent progress in cellular biology and in immunology has led to a paradigm shift in treatment of various challenging diseases, from “one-drug-one-target” approach to combination therapy and multi-targeting drugs approaches. However, although particular drug combinations can theoretically be therapeutically effective in the treatment of various diseases, their clinical success is limited due to dissimilarities in pharmacokinetics and tissue distribution of each drug in the combination.

Different approaches are being developed to overcome these limitations. Bispecific antibodies (bsAbs) are artificial proteins that combine specificities of two antibodies in a single immunoglobulin molecule that simultaneously interferes with multiple receptors or ligands. BsAbs can also place targets into close proximity, either to support protein complex formation on one cell, or to trigger contacts between cells.

Recently, nanoparticles have emerged as a promising platform for the co-delivery of multiple drugs. Zhang, Tian, et al. (Advanced healthcare materials 8.18 (2019): 1900543) provides multitargeted nanoparticles that deliver synergistic drugs across the blood-brain barrier to brain metastases of triple negative breast cancer cells and tumor-associated macrophages.

Dixit et al. (Molecular pharmaceutics 12.9 (2015): 3250-3260) disclosed dual receptor-targeted theragnostic nanoparticles for localized delivery and activation of photodynamic therapy drug in glioblastomas.

U.S. Pat. No. 10,182,986 is directed to methods of delivering a nanoparticle across the blood brain barrier to the brain of a subject by administering to the subject a nanoparticle having a nanoparticle core and a targeting agent.

U.S. Pat. No. 10,478,132-discloses gold nanoparticles coupled with about 4,000-20,000 molecules particles of 2-deoxy-D-glucose, that is capable of binding glucose transporter 1 (GLUT-1), for imaging tumors.

Shilo, Malka, et al. (Nanoscale 6.4 (2014): 2146-2152) developed a technology, which is directed to transport of insulin-targeted gold nanoparticles (INS-GNPs) through the blood-brain barrier for imaging and therapeutic applications.

Trifunctional bispecific antibodies are artificially engineered immunoglobulins that can direct T cells to tumor cells, and also induce recruitment and activation of accessory cells through their Fc region. The simultaneous activation of different mechanisms at the tumor site results in efficient destruction of tumor cells. The binding of the Fc portion of the targeting bispecific antibody to the Fc receptor of antigen presenting cells, dendritic cells and macrophages, results in processing of cancer antigens, presentation of cancer associated peptides to helper T cells and induction of memory T cells. However, developing trifunctional bispecific antibodies is cumbersome, expensive and time consuming and requires long path of regulatory approval.

Human epithelial growth factor receptor 2 (HER2) belongs to the receptor tyrosine kinase family, which consists of four members: HER1 (also known as EGFR), HER2 (also known as Neu), HER3 and HER4. HER2 is a 185-kDa transmembrane glycoprotein containing three components: an extracellular ligand binding domain, a transmembrane domain, and an intracellular domain that has tyrosine kinase activity. Unlike other members, HER2 has no known natural ligand to bind. It exhibits functions through EGFR-HER2 heterodimers, HER2-HER3 heterodimers, and HER2-HER2 homodimers. Amplification of the HER2 gene or overexpression of HER2 receptor plays a crucial role in the cellular transformation, carcinogenesis, and prognosis of many cancer types. HER2-positive tumors account for about 20-30% breast cancer, 20% advanced gastric or gastric or gastro-esophageal junction cancers, 5-15% bladder cancers, 5-15% cervix cancers, 12-15% gallbladder cancers, 8-35% endometrium cancers, 6-7% ovarian cancers, and 15-37% salivary duct cancers. Detection of the expression level of HER2 is conventional and helpful to diagnose, especially in patients with breast cancer which is the most common malignant tumor of women and the cause of death of the second large cancer, and HER2 is considered as an applicable target for antitumor treatment (Yu et al. 2017, Experimental Hematology & Oncology volume 6, 31).

Until now, several HER2-directed therapies have been approved for the HER2-positive breast cancer and non-small cell lung cancer, including trastuzumab, pertuzumab, T-DM1, lapatinib and afatinib (tyrosine kinase inhibitors which blocked EGFR and HER2). HER2-targeted immunotherapy consists of monoclonal antibodies (e.g., trastuzumab, pertuzumab), bispecific antibodies (e.g., MM-111, ertumaxomab) and activated T cells armed with anti-HER2 bispecific antibody (HER2Bi-aATC). Trastuzumab is a classic drug for the treatment of HER2 positive metastatic breast cancer. The combined application of pertuzumab, trastuzumab and paclitaxel has been suggested as a standard therapy for HER2 positive advanced breast cancer. The resistance to anti-HER2 antibody has resulted in disease progression. HER2-directed bispecific antibody may be a promising therapeutic approach for these patients. Ertumaxomab enhanced the interaction of immune effector cells and tumor cells. MM-111 simultaneously binds to HER2 and HER3 and blocks downstream signaling. Besides, HER2Bi-aATC is also an alternative therapeutic approach for HER2 positive cancers.

Trastuzumab, as a classical anti-HER2 antibody, blocked homodimerization of HER2 through binding to the domain IV of HER2. As to pertuzumab, it can prevent the formation of heterodimerization via binding to HER2 subdomain II. Because of the distinct but complementary modes of action, combination of the two agents could strengthen the blockage of downstream signaling, including phosphoinositide 3-kinase/protein kinase B/mammalian target of rapamycin (PI3K/Akt/mTOR) and Ras/Raf/mitogen-activated protein kinase (MAPK). Besides, anti-HER2 monoclonal antibodies could increase endocytosis of HER2 receptor, suppress angiogenesis, and induce tumor cell lysis through antibody-dependent cell-mediated cytotoxicity (ADCC). Synergistic antitumor functions of HER2 antibody with other antitumor agents have been observed in both in vitro and in vivo studies. However, about 70% patients are resistant to trastuzumab, and some exhibited primary resistance. Aimed at the obstacle, researchers have proposed several corresponding strategies: maintaining trastuzumab therapy after progression, combining HER2 inhibitors, and developing novel anti-HER2 monoclonal antibodies. Bispecific antibodies, such as blinatumomab, have achieved great success in hematological malignancies. Among those, HER2-targeted bispecific antibodies which introduced to be widely investigated are also regarded as a remarkable solution.

Ertumaxomab, an intact bispecific antibody, can target HER2 on tumor cells and CD3 on T cells simultaneously, and activate accessory cells via its Fc fragment to exert the function of ADCC. The trifunctional antibody could transiently link immune effector cells to tumor cells and exhibited antitumor activity. MM-111 is another bispecific antibody that specifically targets the HER2/HER3 heterodimer and blocks the binding of heregulin (HRG) and HER3, and then inhibits HER3 downstream signaling pathways. Activated T cell armed with HER2-targeted bispecific antibody (HER2Bi-aATC) exhibited significant inhibition in drug-resistant solid tumors.

There remains an unmet need for multifunctional systems that will enable simultaneous and synchronized delivery of multiple immunoglobulins into tumor tissues and cells to improve the efficacy of cancer treatment.

### SUMMARY OF THE INVENTION

The present invention provides a multifunctional system for the co-delivery of at least two distinct immunoglobulin molecules into target tissues or cells. The present invention further provides methods for preparing the multifunctional system and uses thereof for the treatment of cancer and other diseases. The multifunctional system of the present invention is particularly useful for treatment of tumors located out of the brain or, in some embodiments out of central nervous system (CNS).

The multifunctional system of the invention is based on a core particle which is conjugated to a first and a second immunoglobulin molecules through a first and a second linkers, respectively, and to a transporter or a penetration enhancing moiety through a third linker. Although inorganic core particles are used with added benefit as a simultaneous diagnostic, an organic core is expected to provide a similar effect. The transporter or penetration enhancing moiety facilitates or enhances transport through blood-tumor barrier, for example when configured to treat tumors outside the brain or CNS, facilitates or enhances transport through cancer cell membranes, enables adherence of the delivery system to cancerous cells (e.g., through a specific receptor), and/or improves cancer cell glucose metabolism. Additional active groups, optionally present on the particle surface are capped with a fourth, monofunctional linker.

The immunoglobulin molecules conjugated to the core particle can include various types of antibodies and fragment thereof. In particular, it is herein shown that the combination of two different monoclonal antibodies directed against the human epidermal growth factor receptor 2 (HER2) on a single core particle, which was further conjugated to insulin as a penetration enhancer moiety, is able to inhibit proliferation of cancer cells in vitro and to suppress tumor growth in vivo, in a higher efficiency compared to the efficiency of a mixture of similar particles conjugated separately with each of the antibodies. It is also shown that surprisingly, even in cells expressing low levels of a specific tumor antigen (e.g., HER2), two different antibodies to this antigen, when conjugated to the delivery system, were able to inhibit cancer cell proliferation, with the assistance of insulin that enables binding to the cells via insulin receptor.

Moreover, using two different fluorescently labeled antibodies conjugated to a core particle, it was found that the antibodies underwent co-localization within specific regions. Therefore, the multifunctional delivery system of the invention not only facilitates the tissue penetration of the immunoglobulin molecules, but also confers their synchronized distribution within the tissue or within the tumor. Advantageously, synchronized distribution of different antibodies may significantly improve the therapeutic efficacy of the agents' combination.

The present invention is further based in part on the finding that the GNPs carrying optimized numbers of antibodies per particle have improved anti proliferative activity compared to GNPs with higher or lower density of antibody molecules.

The present invention enables the use of different combinations of antibodies and antibody fragments, as effective tools for anti-cancer immunotherapy against a broad spectrum of malignancies. Antibodies-containing GNPs according to the present invention can bind to cancer cells or to immune cells.

One of the beneficial features of the system of the present invention is that activity of the therapeutic agent, which is conjugated to the delivery system, remains intact, such that it does not necessarily have to be detached from the nanoparticle after delivery to the target tissue, e.g., by using a cleavable linker. Capping moieties, conjugated to remaining potentially active groups on the particle enable desirable distance between the immunoglobulins and other molecules conjugated to the particles.

It was surprisingly found that particles containing a relatively low number of immunoglobulin molecules, in addition to a penetration enhancing moiety, are capable of binding tumor cells and immune cells and eliciting a desired response, such as inhibition of tumor cell proliferation or enhancing apoptosis of tumor cells. In addition, cancer cells which were minimally affected by the bispecific nanoparticles having two immunoglobin molecules, were affected with inclusion of a penetration enhancing moiety on the surface. Surprisingly, breast cancer cells expressing low level of HER2 (hereinafter “HER2-low”) were also affected by particles carrying two different anti HER2 antibodies and insulin as a penetration enhancer.

Another advantage of the delivery system of the present invention is that the specific combination of antibodies is versatile and may be defined for a specific cancer type or for a specific patient based on the tumor antigens of the specific tumor and/or the immune cells' receptors and checkpoint molecules that need to be targeted. The unique linkers of the present invention may be tailored to specifically conjugate the antibodies of interest.

According to one aspect, there is provided a multifunctional particle comprising:
(a) an inorganic particle bound to at least: (i) a first polymeric linker; (ii) a second polymeric linker; (iii) a third polymeric linker; and (iv) a fourth polymeric monofunctional linker;
(b) a first immunoglobulin molecule conjugated to the first linker;
(c) a second immunoglobulin molecule, distinct from the first immunoglobulin molecule, conjugated to the second linker;
(d) a penetration enhancing moiety conjugated to the third linker; and wherein the first and the second immunoglobulin molecules are distinct; and wherein about 2 to 400 immunoglobulin molecules in total are conjugated to each particle, through the first and second linkers.

According to some embodiments, each of the first and second immunoglobulin molecules is conjugated to the first and second polymeric linkers by a covalent bond, which may be identical or different.

According to some embodiments, the first and second polymeric linkers are identical. according to some embodiments, the penetration enhancing moiety is conjugated to the third polymeric linker by a bond selected from a covalent bonds, a semi-covalent bond, and a non-covalent bond.

According to some embodiments, the covalent bond is an amide bond or a disulfide bond.

According to some embodiments, at least one of the linkers is linear.

According to some embodiments, at least one of the linkers is polymeric.

According to some embodiments, the first and the second linkers are linear polymeric linkers.

According to some embodiments, the third linker is a linear polymeric linker.

According to some embodiments, the length of the third linear polymeric linker is substantially different than the lengths of the first and the second linear polymeric linkers.

According to some embodiments, the molecular weight of the third polymeric linker is different than the molecular weight of the first and the second polymeric linkers in at least about 1000 Da.

According to some embodiments, the length of the third linear polymeric linker is substantially higher than the lengths of the first and the second linear polymeric linkers.

According to some embodiments, the first and the second linkers are non-cleavable under physiological conditions.

According to some embodiments, the third linker is non-cleavable under physiological conditions.

According to some embodiments, the third linker is cleavable under physiological conditions.

According to some embodiments, the molecular weight of the first, the second and the third polymeric linkers is within the range of 1,000-10,000 Da. In certain embodiments, the molecular weight of the third polymeric linker is higher than the molecular weights of the first and the second polymeric linkers.

According to some embodiments, the molecular weight of each of the first and the second linear polymeric linkers is within the range of 3500 to 4000 Da, and the wherein the molecular weight of the third linear polymeric linkers is 4500 Da or higher.

According to some embodiments, the third linear polymeric linker is composed of repeating monomer units and at least one of the first and the second linear polymeric linkers is composed of the same repeating monomer units as the third linear polymeric linker, wherein the third linear polymeric linker has a different number of repeating monomer units than the at least one of the first and the second linear polymeric linkers. In certain embodiments, the first, the second and the third linear polymeric linkers are composed of the same repeating monomer units, wherein the third linear polymeric linker has a different number of repeating monomer units than that of the first and the second linear polymeric linkers.

According to some embodiments, the first linker and the second linker are identical.

According to some embodiments, the first and the second linkers are bound to the inorganic particle through a sulfide bond, and the first and the second immunoglobulin molecules are conjugated to the respective linker through an amide bond.

According to some embodiments, each of the first and second immunoglobulin molecules is directed to a tumor-associated antigen (TAA), a tumor-associated receptor, an immune cell receptor or an immune checkpoint protein.

According to some embodiments, the first and the second immunoglobulin molecules are independently selected from the group consisting of an antibody, an antibody fragment comprising at least the antigen binding site, an antibody conjugate and combination thereof.

According to some embodiments, the two different antibodies are capable of binding to the same cancer-specific or cancer-associated cell-surface antigen on a tumor cell.

According to some embodiments, at least one of the antibodies binds to a cancer-specific or cancer-associated cell-surface antigen. According to some specific embodiments, the tumor antigen or tumor-associated antigen is selected from the group consisting of: HER family receptor, EGFR, mesenchymalepithelial transition factor, PSMA, C Nectin-4, CD155, D3, EGFRVIII, Vy9, CD16, CD133, IL-15, and CD19, CD20, CD30, CD38, CD38 and CD138. Each possibility represents a separate embodiment of the invention.

According to some specific embodiments, the two antibodies bind to Herceptin family receptor. According to some specific embodiments, the two antibodies bind to HER2.

According to some embodiments, each of the two different antibodies is capable of binding to a different cancer-specific or cancer-associated cell-surface antigen on a tumor cell.

According to some specific embodiments, one antibody binds to HER2 and one to HER3.

According to some embodiments, the GNPs comprises a first antibody capable of binding to immune cells (e.g., T cells or NK cells), and a second antibody capable of binding to at least one cancer-specific or cancer-associated cell-surface antigen on a tumor cell.

According to some embodiments, one of the antibodies is specific to an NK receptors selected from the group consisting of: a Natural Cytotoxicity Receptors (NCR), NKp30, NKp44, NKp46, CD16, CD314, and CD94/NKG2C. Each possibility represents a separate embodiment of the invention.

According to some specific embodiments, at least one of the antibodies is specific to a checkpoint molecule. According to yet other embodiments, the antibody is specific to a checkpoint molecule selected from the group consisting of PD-1, PD-L1, CTLA-4, 4-1BB, OX40, TIM3, TIGIT, LAG-3, and CD47. Each possibility represents a separate embodiment of the invention.

Non limiting examples for targets of the antibodies of the multifunctional GNPs of the present invention include: HER family receptors, EGFR, mesenchymalepithelial transition factor, PSMA, Nectin-4, CD155, CEA, CD3, EpCam, EGFRVIII, Vy9, CD16, CD133, IL-15, CD19, CD20, CD30, CD38, CD38 and CD138, IGF-1 and IGF-2, VEGF, Ang2, cMET, DLL4, CD137, IGF-R1, PMEL, B7H3, GPA33, GPC3, PD-1, PD-L1, CTLA-4, 4-1BB, OX40, TIM3, TIGIT, LAG-3, and CD47, NCRs, NKp30, NKp44, NKp46, CD16, CD314, and CD94/NKG2C.

According to some embodiments, at least one of the antibodies binds to PD-1.

According to some embodiments, at least one of the antibodies binds to PD-L1.

According to some specific embodiments, each multifunctional GNP is conjugated with a pair of antibodies targeting antigens selected from the group consisting of: HER2 and HER2, HER2 and HER3, HER2 and PD-1, HER2 and CTLA-4, PD-1 and PD-L1, PD-1 and CTLA-4, CEA and CD3, PSMA and CD3, EGFRvIII and CD3, EpCam and CD3, HER2 and Vy9, CD16 and CD133, CD16 and IL-15, CD15 and CD19, CD16 and CD133, IGF-1 and IGF-2, VEGF and Ang2, EGFR and cMET, DLL4 and VEGF, HER2 and CD3, PD-1 and LAG3, PD-L1 and CD137, PSMA and CD3, IGF-R1 and HER3, PMEL and CD3, B7H3 and CD3, GPA33 and CD3, GPC3 and CD3. Each possibility represents a separate embodiment of the invention.

According to some embodiments, one of the antibodies binds to PD-1 and the other binds to PD-L1.

According to some embodiments, at least one of the antibodies is capable of binding to T cells, NK cells, dendritic cells, or macrophages or to an Fc receptor on activated immune cells.

According to some embodiments, the immune cells are selected from the group consisting of NK cells, T cells, NKT cells, macrophages, and any combination thereof.

According to some embodiments, the antibody is a monoclonal antibody.

According to some embodiments, the GNPs comprise at least one antibody selected from the group consisting of non-human, chimeric, humanized, human antibody, and any combination thereof.

According to some specific embodiments, the antibody is a chimeric monoclonal antibody.

According to some embodiments, the chimeric antibody comprises a human-derived constant region selected from the group consisting of: IgG1, IgG2, IgG3, and IgG4.

According to some embodiments, at least one antibody is a humanized antibodies.

According to some embodiments, about 2 to 40 antibodies are conjugated, through a linker, to each particle. According to some embodiments, about 2 to 20 antibody molecules are conjugated, through a linker, to each particle. According to some embodiments, about 2 to 10 antibody molecules are conjugated, through a linker, to each particle. According to yet other embodiments, about 4 to 40 antibody molecules are conjugated, through a linker, to each particle. According to yet other embodiments, about 5 to 30 antibody molecules are conjugated, through a linker, to each particle. According to some specific embodiments, about 10 to 20 or about 15 to 25 antibody molecules are conjugated, through a linker, to each particle.

According to some embodiments, about 20 to 400 antibody fragments are conjugated to each particle, through a linker. According to yet other embodiments, about 40 to 400 antibody fragments are conjugated to each particle, through a linker. According to yet other embodiments, about 50 to 300 antibody fragments are conjugated to each particle, through a linker. According to some specific embodiments, about 100 to 200 or about 50 to 350 antibody molecules are conjugated to each particle through a linker.

According to some embodiments, the third linear polymeric linker constitutes from about 10% mol to 40% mol of the total polymeric linkers bound to the inorganic particle.

According to some embodiments, each one of the first and the second linear polymeric linkers independently constitutes from about 5% mol to 40% mol of the total polymeric linkers bound to the inorganic particle.

According to some embodiments, the first, the second and the third linear polymeric linkers independently comprise a polymer selected from the group consisting of: a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives, and combinations thereof. According to certain embodiments, at least one of the first, the second and the third linear polymeric linkers is a polyether. In some exemplary embodiments, the polyether is polyethylene glycol (PEG). The polyethylene glycol can be selected from a thiolated PEG acid (HS-PEG-COOH) and a thiolated PEG amine (HS-PEG-NH₂), wherein the thiolated end is bound to the inorganic particle and the acid or amine end is conjugated to the respective immunoglobulin molecule or to the penetration enhancing moiety.

According to some embodiments, the multifunctional particle further comprises a non-functional capping moiety bound to the inorganic particle directly or through a linker or a spacer. According to some embodiments, the linker is a monofunctional polymeric linker to which the capping moiety is attached. According to some embodiments, said polymeric linker comprises a polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives, and combinations thereof. In some exemplary embodiments, said fourth polymeric linker comprises a polyether, wherein the polyether is methoxy polyethylene glycol (mPEG).

According to some embodiments, the inorganic particle is a nanoparticle selected from the group consisting of a metal nanoparticle, a metal oxide nanoparticle, a ceramic nanoparticle, and any combination thereof. The metal can be selected from the group consisting of gold, silver, platinum, iron, and any combination thereof. The metal oxide can be selected from the group consisting of iron oxide, magnesium oxide, nickel oxide, cobalt oxide, aluminum oxide, zinc oxide, copper oxide, manganese oxide, and any combination thereof. In some specific embodiments, the inorganic particle is selected from the group consisting of a gold nanoparticle, an iron (III) oxide nanoparticle, and an iron (II,III) oxide nanoparticle. In further specific embodiments, the inorganic particle is a gold nanoparticle.

According to some embodiments, the inorganic particle is a nanoparticle having a diameter of 10-160 nm.

According to some embodiments, the moiety connected to the third linker is a molecule that generally relates to sugar metabolism. According to some embodiments, the moiety connected to the third linker is a penetration enhancer or transporter.

According to some embodiments, the moiety connected to the third linker comprises a molecule that facilitates or enhances glucose entry into cells or cell metabolism.

According to some embodiments, the penetration enhancing moiety is capable of increasing tumor cell metabolism. According to some embodiments, the penetration enhancing moiety is selected from the group consisting of: insulin, an antibody specific for an insulin receptor, a polypeptide that specifically binds to the insulin receptor, insulin-like growth factor 1, an antibody specific for an insulin-like growth factor receptor 1, a polypeptide that specifically binds to the insulin-like growth factor receptor 1, a cell-penetrating peptide (CPP), and any combination thereof. In certain embodiments, the moiety is selected from insulin and glucose. According to some embodiments, the glucose is 2-deoxy-D-glucose.

According to some embodiments, the penetration enhancing moiety actively enhances penetration through the blood-tumor barrier.

According to some embodiments, the multifunctional particle further comprises an additional immunoglobulin conjugated via a linker or a spacer to the inorganic particle.

According to some exemplary embodiments, the inorganic particle is a gold nanoparticle, the first linear polymeric linker and the second linear polymeric linker are each independently a thiolated PEG3500 acid or thiolated PEG3500 amine, the third linear polymeric linker is a thiolated PEG5000 acid or thiolated PEG5000 amine, and the penetration enhancing moiety is insulin.

According to some exemplary embodiments, the inorganic particle is a gold nanoparticle, the first linear polymeric linker is a thiolated PEG1000 acid or thiolated PEG1000 amine, the second linear polymeric linker is a thiolated PEG3500 acid or thiolated PEG3500 amine, the third linear polymeric linker is a thiolated PEG5000 acid or thiolated PEG5000 amine, and the penetration enhancing moiety is insulin.

According to another aspect, there is provided a process for preparation of the multifunctional particle according to the various embodiments described hereinabove. According to some embodiments, the process comprises sequential steps of: (a) partially coating a surface of an inorganic particle with a first linear polymeric linker followed by conjugating the first linear polymeric linker to a first biologically active molecule; (b) partially coating the surface of the inorganic particle with a second linear polymeric linker followed by conjugating said second linear polymeric linker to a second biologically active molecule; and (c) partially coating the surface of the inorganic particle with a third linear polymeric linker followed by conjugating said third linear polymeric linker to a penetration enhancing moiety, wherein steps (a), (b) and (c) can be performed in any order.

According to some embodiments, the a process for preparation of a multifunctional particle, comprises the sequential steps of: (a) partially coating a surface of an inorganic particle with a first linear polymeric linker and a second linear polymeric linker, followed by conjugating the first and the second linear polymeric linkers to a first biologically active molecule and a second biologically active molecule, wherein the first linear polymeric linker and the second linear polymeric linker are identical and wherein the first biologically active molecule is distinct from the second biologically active molecule; and (b) partially coating the surface of the inorganic particle with a third linear polymeric linker followed by conjugating the third linear polymeric linker to a penetration enhancing moiety, wherein the length of the third linear polymeric linker is substantially different than the length of the first and the second linear polymeric linkers, wherein the molecular weight of the third polymeric linker is different than the molecular weight of the first and the second polymeric linkers in at least about 1000 Da, and wherein step (a) and step (b) can be performed in any order.

According to some embodiments, the particle is a gold nanoparticle (GNP) and the process for the preparation of multifunctional gold nanoparticles, comprises the sequential steps of: (a) reduction of HAuCl₄; (b) simultaneous incubation of the reduced GNPs with one monofunctional linker and two different heterofunctional linkers; (c) activation the GNPs to obtain free COOH groups; (d) conjugation of the penetration enhancing moiety: (d) conjugation of the two different antibodies by incubating with a solution comprising their mixture.

According to some embodiments, analysis of the GNPs is performed following each step using methods known in the art.

According to some embodiments, the monofunctional linker is mPEG-SH. According to a specific embodiment, the monofunctional linker is mPEG5000-SH or mPEG6000-SH, and it is added to cover about 60-95% of particle surface.

According to some embodiments, the heterofunctional likers are COOH-PEG-SH. According to some embodiments, one heterofunctional liker is COOH-PEG5000-SH and it is added in a concentration to cover about 15% of particle surface. According to some embodiments, the other heterofunctional liker is COOH-PEG3500-SH and it is added in a concentration to cover about 5% of particle surface.

According to some embodiments, activation of the terminated acid PEGs (linkers) to conjugate the immunoglobulin molecules is performed by mixing the GNPs with (1-ethyl-3-(3-dimethylaminopropyl) carbodiimide HCl (EDC) and/or sulfo-NHS.

According to some embodiments, the penetration enhancing moiety is insulin, and its conjugation is performed by incubating for 1-5 hours with the activated GNPs, at a concentration of about 50-500 IU/ml.

According to some embodiments, the antibodies are two different antibodies against human HER2. According to some embodiments, the two different antibodies against HER2 are trastuzumab and pertuzumab. According to some embodiments, the two different antibodies against HER2 are trastuzumab and pertuzumab and the first and second polymer linker are greater than 3500 Da. According to some embodiments, the two antibodies are incubated overnight at a concentration of 1-50 mg/ml, with activated GNPs that are conjugated with a penetration enhancing moiety.

According to some embodiments, the analysis of GNPs is performed using Dynamic Light Scattering (DLS).

According to some embodiments, quantification of the antibodies and the penetration enhancing moiety (e.g., insulin) attached to the PEG groups on the GNPs is performed by enzyme-linked immunosorbent assay (ELISA) of the supernatants containing the unbound proteins left after precipitation by centrifugation of the GNPs.

According to some embodiments, the first polymeric linker has a first functional end group configured to bind the first biologically active molecule; the second polymeric linker has a second functional end group configured to bind the second biologically active molecule; and the third polymeric linker has a third functional end group configured to bind a penetration enhancing moiety, wherein at least two of the first, the second and the third functional end groups are identical.

According to some embodiments, the process further comprises partially coating the surface of the inorganic particle with a capping moiety, optionally connected through a fourth polymeric linker, wherein said fourth polymeric linker is a monofunctional linker.

According to some embodiments, each one of the first linear polymeric linker and the second linear polymeric linker is added in an amount suitable for covering between 5% and 40% of the surface of the inorganic particle, and the third linear polymeric linker, if added is in an amount suitable for covering between 5% and 40% of the surface of the inorganic particle. According to some embodiments, a capping moiety is used to cover about 20-85% of the surface of the inorganic particle. According to some embodiments, the capping moiety comprises a methyl group attached to a linker. According to some embodiments, the capping moiety comprises a methyl group attached to a PEG linker.

The multifunctional GNPs according to the invention are provided, according to some embodiments, for treatment of cancer.

According to some embodiments, the multifunctional GNPs are for use in elimination or inhibition of cancer progression, metastatic spread.

According to yet another aspect, there is provided a pharmaceutical composition comprising the multifunctional particle according to the various embodiments presented hereinabove and a pharmaceutically acceptable carrier or excipient.

According to some embodiments, the pharmaceutical composition is provided for use in the treatment, and/or monitoring cancer or a tumor located outside the brain of a subject in need thereof. According to some embodiments, the pharmaceutical compositions are used in inhibiting or eliminating cancer progression or for preventing or inhibiting formation or spread of cancer metastases.

According to some embodiments, the solid tumor or tumor metastases are located outside the CNS.

According to some specific embodiments, the solid tumor is selected from the group consisting of breast, lung, bladder, pancreatic and ovarian.

According to some embodiments of the invention, the cancer is selected from the group consisting of a lung cancer, a breast cancer, a colorectal cancer, a melanoma, an ovarian cancer, a pancreatic cancer, a colon cancer, a cervical cancer, a kidney cancer, a thyroid cancer, a prostate cancer, a renal cancer, a throat cancer, a laryngeal carcinoma, a bladder cancer, a hepatic cancer, a fibrosarcoma, an endometrial cells cancer, a glioblastoma, sarcoma. Each possibility represents a separate embodiment of the invention.

According to certain embodiments, the cancer is selected from the group consisting of: breast cancer, colorectal cancer, lung cancer, kidney cancer, melanoma, and prostate cancer. Each possibility represents a separate embodiment of the invention.

According to other embodiments, the solid tumor is a breast adenocarcinoma, or the metastases are derived from a primary breast adenocarcinoma.

According to some embodiments, the breast cancer is a metastatic breast cancer.

According to some embodiments, the breast cancer is a positive for HER2.

According to some embodiments, the breast cancer is negative for HER2. According to some embodiments, the breast cancer is characterized as HER2-low.

According to some embodiments, the breast cancer is a triple negative breast cancer (TNBC).

The present invention also provides, according to some embodiments, a pharmaceutical composition comprising a pharmaceutically acceptable carrier or excipient and a multifunctional particle comprising:
(a) an inorganic particle bound to at least: (i) a first linear polymeric linker; (ii) a second linear polymeric linker; (iii) a third linear polymeric linker; and (iv) a fourth polymeric monofunctional linker;
(b) a first immunoglobulin molecule conjugated to the first linear polymeric linker;
(c) a second immunoglobulin molecule, covalently conjugated to the second linear polymeric linker; and
(d) a penetration enhancing moiety conjugated to the third linear polymeric linker;
   wherein the first and the second immunoglobulin molecules are distinct; for use in treating or monitoring a primary tumor or metastases located outside the brain.

According to some embodiments, a pharmaceutical composition comprising a multifunctional particle is provided for use in treating or monitoring a primary tumor or metastases located outside the brain, wherein the multifunctional particle comprises:
(a) an inorganic particle bound to at least: (i) a first linear hetrofunctional polymeric linker; (ii) a second linear hetrofunctional polymeric linker; (iii) a third linear hetrofunctional polymeric linker; and (iv) a fourth polymeric monofunctional linker;
(b) a first immunoglobulin molecule covalently conjugated to the first linear polymeric linker;
(c) a second immunoglobulin molecule, covalently conjugated to the second linear polymeric linker; and
(d) a penetration enhancing moiety conjugated to the third linear polymeric linker;
   wherein the first and the second immunoglobulin molecules are distinct; and wherein about 2 to 400 immunoglobulin molecules in total, are conjugated to each particle, through the first and second linkers.

According to some embodiments, the pharmaceutical composition is formulated for at least one of an intravenous (IV) administration, an intranasal (IN) administration, and intraperitoneal (IP) administration.

According to some embodiments, the pharmaceutical composition described herein is administered as part of a regiment of cancer treatment selected from the group consisting of chemotherapy, immunotherapy, biotherapy, hormonal therapy, radiation therapy, bone-marrow transplantation, surgery, and any combination thereof.

According to some embodiments, the pharmaceutical composition according to the present invention is for use in cancer immunotherapy or in enhancing immune responses.

According to some embodiments, the pharmaceutical composition further comprises killer cells (e.g., T cells, NK cells, NKT cells and/or macrophages).

The present invention provides according to another aspect a method for treating a subject having a cancer or a tumor located outside the brain, the method comprising administering to the subject a pharmaceutical composition comprising the multifunctional GNPs disclosed herein.

According to some embodiments, the method for treating a subject having a cancer or a tumor located outside the brain, comprises administering to the subject a pharmaceutical composition comprising a multifunctional particle, wherein the multifunctional particle comprises:
(a) an inorganic particle bound to at least: (i) a first linear polymeric linker; (ii) a second linear polymeric linker; (iii) a third linear polymeric linker; and (iv) a fourth polymeric monofunctional linker;
(b) a first immunoglobulin molecule conjugated to the first linear polymeric linker;
(c) a second immunoglobulin molecule, covalently conjugated to the second linear polymeric linker; and
(d) a penetration enhancing moiety conjugated to the third linear polymeric linker;
   wherein the first and the second immunoglobulin molecules are distinct.

According to some embodiments, the treatment results in a decrease in tumor size or in the number of metastases in the subject.

According to some embodiments, the tumor or metastases are located outside the CNS.

According to certain embodiments, the method comprises administering multifunctional GNPs comprising at least one antibody that targets Her2/neu protein.

According to some embodiments, the method comprises administering multifunctional GNPs comprising two different antibodies that targets Her2/neu, wherein each GNP is conjugated with the two different antibodies to HER2.

The methods of the present invention include stand-alone treatments as well as combination with any anti-cancer treatment.

According to a specific embodiment, the method comprises administration of a pharmaceutical composition comprising at least one multifunctional GNP to a subject in need thereof, and administration of at least one anti-cancer agent. Such administration may be performed simultaneously or at separate times.

Any tumor characterized by expressing a specific tumor antigen or tumor-associated antigen, and any cancer eligible for T-cell therapy, may be treatable with the multifunctional GNPs of the present invention.

According to some embodiments, the cancer is a solid cancer or comprises a solid tumor located outside the brain.

According to some embodiments, the solid tumor is a metastatic solid tumor.

According to some embodiments, the solid tumor is a primary resistant solid tumor or tumor metastases located outside the brain.

According to some embodiments, the solid tumor or tumor metastases are located outside the CNS.

According to some specific embodiments, the solid tumor is selected from the group consisting of breast, lung, bladder, pancreatic and ovarian.

According to some embodiments of the invention, the cancer is selected from the group consisting of a lung cancer, a breast cancer, a colorectal cancer, a melanoma, an ovarian cancer, a pancreatic cancer, a colon cancer, a cervical cancer, a kidney cancer, a thyroid cancer, a prostate cancer, a renal cancer, a throat cancer, a laryngeal carcinoma, a bladder cancer, a hepatic cancer, a fibrosarcoma, an endometrial cells cancer, a glioblastoma, sarcoma. Each possibility represents a separate embodiment of the invention.

According to certain embodiments, the cancer is selected from the group consisting of: breast cancer, colorectal cancer, lung cancer, kidney cancer, melanoma, and prostate cancer. Each possibility represents a separate embodiment of the invention.

According to other embodiments, the solid tumor is a breast adenocarcinoma, or the metastases are derived from a primary breast adenocarcinoma.

According to some embodiments, the breast cancer is a metastatic breast cancer.

According to some embodiments, the breast cancer is a positive for HER2.

According to some embodiments, the breast cancer is negative for HER2. According to some embodiments, the breast cancer is characterized as HER2-low.

According to some embodiments, the breast cancer is a triple negative breast cancer (TNBC).

According to a yet further aspect, the present invention provides a method of immunotherapy of cancer comprising administering multifunctional GNPs that blocks negative regulators such as checkpoint inhibitors or regulatory T cell inhibitors. According to some embodiments, the checkpoint inhibitor is selected from CTLA-4 and PD-1/PD-L1.

According to some embodiments, the multifunctional GNPs are administered intravenously or inside a tumor.

According to some embodiments, the method is part of a treatment regimen comprising an additional cancer treatment. According to some embodiments, the additional cancer treatment is selected from the group consisting of chemotherapy, immunotherapy, biotherapy, hormonal therapy, radiation therapy, bone-marrow transplantation, surgery, and any combination thereof.

According to an additional aspect, the present invention provides a method for enhancing immune response in a subject in need thereof comprising administering to said subject a pharmaceutical composition as described herein.

According to some embodiments, the method of treating cancer comprises administering or performing at least one additional anti-cancer therapy. According to certain embodiments, the additional anticancer therapy is surgery, chemotherapy, radiotherapy, or immunotherapy. In specific embodiments, the additional therapy is radiation therapy.

According to some embodiments, the method of treating cancer comprises administration of the antibody and an additional anti-cancer agent. According to some embodiments, the additional anti-cancer agent is selected from the group consisting of: immune-modulator, agent that inhibits immune co-inhibitory receptor, activated lymphocyte cells, kinase inhibitor, and chemotherapeutic agent.

According to some embodiments, the additional immune-modulator is an antibody against an immune checkpoint molecule. According to some embodiments, the additional immune modulator is an antibody against an immune checkpoint molecule selected from the group consisting of human programmed cell death protein 1 (PD-1), PD-L1 and PD-L2, carcinoembryonic antigen-related cell adhesion molecule 1 (CEACAMI), lymphocyte activation gene 3 (LAG3), CD137, OX40 (also referred to as CD134), killer cell immunoglobulin-like receptors (KIR), TIGIT, PVR, CTLA-4, NKG2A, GITR, and any other checkpoint molecule or a combination thereof. Each possibility represents a separate embodiment of the invention.

According to some embodiments, the anti-cancer agent is selected from the group consisting of: Erbitux, cytarabine, fludarabine, fluorouracil, mercaptopurine, methotrexate, thioguanine, gemcitabine, vincristine, vinblastine, vinorelbine, carmustine, lomustine, chlorambucil, cyclophosphamide, cisplatin, carboplatin, ifosfamide, mechlorethamine, melphalan, thiotepa, dacarbazine, bleomycin, dactinomycin, daunorubicin, doxorubicin, idarubicin, mitomycin, mitoxantrone, plicamycin, etoposide, teniposide and any combination thereof. Each possibility represents a separate embodiment of the invention.

According to some embodiments of the invention, the subject is a human subject.

According to some embodiments, the method of treating cancer involves preventing or reducing formation, growth or spread of metastases in a subject.

According to an additional aspect, there is provided a method for a simultaneous delivery of at least two antibodies to a specific tissue, body region, to cancerous cells or specific spatial area of a tumor of a subject, the method comprising administering to the subject the pharmaceutical composition according to the various embodiments presented hereinabove. According to some embodiments, upon administration, the at least two antibodies exhibit synchronized distribution within the target tissue or body region or to cancerous cells.

According to some embodiments, the pharmaceutical composition is administered to the subject by at least one of an oral, intravenous (IV), an intranasal (IN), and an intraperitoneal (IP) administration.

According to some embodiments, the method further comprises a step of imaging a tissue or a body region or to cancerous cells of the subject to thereby evaluate accumulation of the multifunctional particle in the of said subject. The imaging can be performed using an imaging system selected from the group consisting of computed tomography imaging (CT), X-ray imaging, magnetic resonance imaging (MRI), positron emission tomography (PET), single-photon emission computed tomography (SPECT), ultrasound (US), and any combination thereof.

According to some embodiments, the body region is a breast or the breasts.

According to some embodiments, the cancer located outside the brain is a primary cancer or secondary cancer; the inorganic particle is a radiosensitizer; and the method further comprises radiation therapy.

According to an aspect, the present invention provides a method of diagnosing or prognosing cancer in a subject, the method comprising determining the expression level of a tumor antigen or an immune cell receptor in a biological sample of said subject using the multifunctional GNPs of the present invention.

A kit for measuring the expression or presence of a tumor antigen or an immune cell receptor in biological sample is also provided multifunctional GNPs according to the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 represents a schematic illustration of a gold nanoparticle (GNP; 1) bound to: (i) a first polymeric linker (2) which is conjugated to an insulin (4); (ii) a second polymeric linker (3) which is conjugated to a first antibody (5) and to a second antibody (6); and (iii) a capping polymer moiety (7).

FIG. 2 displays the in vivo effect of dual-antibody GNPs on HER2 positive cancer cells. GNPs conjugated with insulin and two different anti-HER2 antibodies were tested in the HER2 positive breast cancer cell line BT474, in comparison to a mixture of GNPs conjugated to insulin and a single antibody. Untreated cells served as control. Cells were incubated for 5 days with the following conditions: 1. Control-untreated, 2. Mixture of GNPs with Trastuzumab & GNPs with Pertuzumab, 3. Bi-specific GNPs with both Trastuzumab & Pertuzumab conjugated to the same particle.

FIG. 3A displays the in vitro effect of the bi-specific nanocomplex on human breast cancer cell line BT474 expressing high level of HER2 receptors. Cells were incubated for 5 days with the following conditions: (i) Control-untreated; (ii) Bi-specific GNPs with both Trastuzumab & Pertuzumab anti HER2 antibodies conjugated to the same particle without a penetration enhancing molecule; and (iii) Bi-specific GNPs with both Trastuzumab & Pertuzumab conjugated to the same particle in addition to insulin molecules as a penetration enhancer.

FIG. 3B displays the in vitro effect of the bi-specific nanocomplex on human breast cancer cell line MCF7, expressing relatively low level of HER2 receptors. Cells were incubated for 5 days with the following conditions: (i) Control-untreated; (ii) Bi-specific GNPs with both Trastuzumab & Pertuzumab anti HER2 antibodies conjugated to the same particle without a penetration enhancing molecule; and (iii) Bi-specific GNPs with both Trastuzumab & Pertuzumab conjugated to the same particle in addition to insulin molecules as penetration enhancers.

FIG. 4 demonstrates the results of determining the optimal density, e.g., number of antibody molecules, per particle. Different compositions of the nanocomplex with increasing number of attached antibodies were tested in vitro, for inhibition of proliferation of BT474 human breast cancer cells that overexpress HER2 receptors. Cells were incubated for 5 days with the following conditions: (i) Control-untreated; (ii) GNPs with 2 antibody molecules attached to each particle; (iii) GNPs with 11 antibody molecules attached to each particle; (iv) GNPs with 18 antibody molecules attached to each particle; (v) GNPs with 20 antibody molecules attached to each particle and (vi) GNPs with 30 antibody molecules attached to each particle.

FIG. 5 depicts inhibition of tumor growth in vivo using GNPs conjugated with the anti HER2 antibodies Trastuzumab and Pertuzumab and insulin as a penetration enhancer. Breast cancer cells BT474 were administered subcutaneously to mice and after 2 weeks the mice were injected IP once a week, for 4 consecutive weeks. The treatment groups tested were: a control group, a group that received the mixture of the free antibodies, a group that received a mixture of GNPs one with Trastuzumab and others with Pertuzumab, and the fourth group received bi-functional GNPs (with both antibodies conjugated to the same particle).

FIG. 6 demonstrates the utility of the multifunctional system in immunotherapy by simultaneous delivering on GNPs, anti PD-1 that targets receptors on T-cells, and anti PD-L1 that binds receptors on the tumor cells. Peripheral blood mononuclear cells (PBMCs) were activated by anti CD3 and anti CD28 and then incubated for 6 days with a mixture of free anti PD-1 and anti PD-L1 antibodies; or with GNPs conjugated with both anti PD-1 and anti PD-L1 antibodies. After 6 days, medium was removed to detect cytokines and PBMCs were collected and added to H1299 lung cancer cells and incubated for 18 hours to determine T-cell cytotoxicity on the target cells. Cell proliferation was measured by ELISA.

### DETAILED DESCRIPTION OF THE INVENTION

The present invention provides a platform for the synchronized delivery of combinations of distinct immunoglobulins to a specific tissue, cancerous cells or body region, a process of preparation of the system, pharmaceutical compositions comprising said system, and uses thereof for therapeutic and diagnostic applications. In particular, the present invention provides a multifunctional system for delivery of antibodies into a malignancy or into cancer cells, or to a specific spatial area of a tumor and/or for recruiting immune cells, such as killer cells, to cancer cells outside the brain or outside the CNS.

The multifunctional delivery system of the present invention is based on a core particle which is conjugated through a first polymeric linker to a first immunoglobulin; through a second polymeric linker to a second, distinct immunoglobulin; and through a third polymeric linker to a penetration enhancing moiety, which facilitates or enhances transport through blood-tumor barrier or may be, according to some embodiments, a cell-internalizing transporter or enhancer, such as a cell metabolism enhancer that facilitates of increases glucose consumption by the cells, in particular by cancer cells. Each one of the first and the second immunoglobulin molecules can be an intact antibody, or an antibody fragment or construct, comprising at least the antigen binding site. Without wishing to be bound by any theory or mechanism, it is hypothesized that the transporter promotes the penetration of the conjugated particle through blood-tumor barrier and/or through other biological membranes and that a cell metabolism enhancer, promotes the penetration of the entire conjugated system through a receptor such as an insulin receptor.

Advantageously, by delivering simultaneously on a single vehicle, two or more distinct immunoglobulins having different cellular targets and/or different mechanism of action (e.g., tumor receptors and immune cell activators), an improved therapeutic and/or diagnostic efficacy can be achieved. Thus, the delivery system of the invention can be useful for the treatment and/or diagnosis of a wide range of conditions, particularly malignancies.

The present invention is based in part on the surprising finding that two different anti HER2 antibodies on a single core particle, which was further conjugated to insulin as a penetration enhancer moiety, is able to inhibit proliferation of cancer cells in vitro and to suppress tumor growth in vivo, in a higher efficiency compared to the efficiency of a mixture of similar particles conjugated separately with each of the antibodies. This was also shown for a combination of anti PD-1 and anti PD-L1 antibodies.

According to the principles of the present invention, the first and the second immunoglobulins are conjugated to the external surface of the core particle through polymeric linkers, rather than being loaded or encapsulated within the particle core. Importantly, the activity of the immunoglobulin molecules is maintained despite this conjugation to the core particle, so that it is not necessarily required to release said agents from the system upon penetration to the malignant cell or tissue.

As a diagnostic, this approach enables, in some embodiments, early and accurate detection of certain malignancies and other diseases or disorders. For example, when the multifunctional particle comprises two or more immunoglobulin molecules that target the system to diseased/damaged cells, and the core particle is an imaging agent that enables tracking the particles in vivo using a suitable imaging modality.

As a therapeutic, in some embodiments, this approach enables the delivery of efficient therapeutic combinations. In some embodiments, the combination of distinct therapeutic immunoglobulins on a single platform results in optimized synergism of the agents combination.

In particular, this approach is suitable for treatment of cancer, using combination of different binding moieties directed toward tumor antigen/s and/or immune cells.

In some embodiments, a combined therapeutic and diagnostic use is enabled, e.g., by using therapeutic active immunoglobulins, such as antibodies, that are conjugated to a core particle which constitutes an imaging contrast agent, e.g., gold nanoparticle.

### Multifunctional System

According to one aspect, there is provided a multifunctional system for the simultaneous delivery of distinct immunoglobulin to tissues and cells outside the brain, the multifunctional system comprises:
(a) a core particle bound to at least: (i) a first polymeric linker; (ii) a second polymeric linker; and (iii) a third polymeric linker;
(b) a first immunoglobulin molecule conjugated to the first polymeric linker;
(c) a second immunoglobulin molecule. Distinct from the first immunoglobulin, conjugated to the second polymeric linker; and
(d) a penetration enhancing moiety conjugated to the third polymeric linker.

According to another aspect, there is provided a multifunctional system comprising:
(a) a core particle bound to at least: (i) a first polymeric linker; (ii) a second polymeric linker; and (iii) a third polymeric linker; and
(b) a penetration enhancing moiety conjugated to the third polymeric linker, wherein each of the first and the second polymeric linkers has a free functional end group configured for conjugating a first immunoglobulin and a second, distinct immunoglobulin molecules.

In some embodiments, the length of the third polymeric linker is substantially different than the length of at least one of the first and the second polymeric linkers. In some embodiments, the length of the third polymeric linker is substantially higher than the length of at least one of the first and the second polymeric linkers.

The term “multifunctional system”, which can be used herein interchangeably with the terms “multifunctional particle”, “multifunctional GNP”, and “co-delivery system”, refers to a system that is capable of accomplishing at least two objectives or is capable of performing a single advanced function through incorporation of at least two functional units. The system of the invention incorporates multiple functional units having distinct objectives, including at least the first and the second immunoglobulin molecules which have distinct binding site and, in some embodiments, also distinct targets and/or distinct activity, and a penetration enhancing moiety, e.g., an internalizing transporter moiety which assists in delivering the system across biological membranes, for example, blood-tumor barrier.

As used herein, the term “co-delivery” can be interchangeably used with the term “simultaneous delivery” and means that the two distinct immunoglobulins are delivered simultaneously in a single composition to their target, e.g., into a tumor area, cancerous cells or to specific region or tissue in the body of a subject that is outside the brain and in some embodiments outside the CNS. In some embodiment, “co-delivery” means synchronized delivery, i.e., that upon administration, the distinct active agents exhibit synchronized pharmacokinetics and biodistribution. In some related embodiments, the two immunoglobulins exhibit synchronized distribution within a body tissue or region, or within a solid tumor or tumor metastases. The term “synchronized distribution” as used herein means that the two active agents co-localize within the same regions, tissues, population of cells or tumors. In some embodiments, the two immunoglobulins accumulate in the same malignancy or in a specific spatial area of a tumor of a subject. In yet other embodiments, the two immunoglobulins bring two different types of cells, to a closer proximity. In some embodiments, the different types of cells comprise cancer cells and immune cells.

In some embodiments, the synchronized pharmacokinetics and biodistribution results in optimized synergism of the agents combination. In particular relevant embodiments, one of the immunoglobulins is a monoclonal antibody directed to a tumor antigen and the second immunoglobulin is a monoclonal antibody that activates an immune cell. According to some embodiments, the two active agents are antibodies against same or different tumor antigens.

The terms “delivery” and “delivered” encompass both delivery of the immunoglobulin(s) by releasing said active agent(s) from the delivery system (e.g., by using cleavable linkers), and delivery of the immunoglobulin(s) while being conjugated (e.g., by covalent conjugation) to the delivery system. Advantageously, the composition of the multifunctional system of the invention does not interfere with the functionality of the immunoglobulins, such that their release from the system is not necessarily required.

The term “distinct” as used herein means that the first immunoglobulin molecule is distinguishably different than the second immunoglobulin molecule. It is to be understood that the term “distinct” encompass also different molecules of the same type, e.g., two antibodies having different specificities. It is further to be understood that the term “distinct” also encompass different molecules that comprise a similar fragment. For Example, a whole antibody (e.g., IgG) and a fragment of said antibody (e.g., Fc/Fab region or a scFv) are considered as distinct active agents.

As used herein, the term “core particle” refer to a particle which constitutes the central part of the co-delivery system. In some embodiments, the core particle is a nanoparticle. The term “nanoparticle” refers to a particle having a diameter of between 1 to 1000 nm.

In some embodiments, the core particle is selected from the group consisting of a metal particle, a metal oxide particle, a metal carbide particle, a lipid particle, a carbon-based particle, a ceramic particle, a polymeric particle and a liposome. Each possibility represents a separate embodiment of the present invention. In some embodiments, the core particle is an inorganic particle. In some embodiments, the inorganic particle is selected from the group consisting of a metal particle, a metal oxide particle and a ceramic particle. In some embodiments, the inorganic particle is selected from the group consisting of a metal particle and a metal oxide particle. In some embodiments, the inorganic particle is metal particle. In other embodiments, the inorganic particle is a metal oxide particle. In specific embodiments, the inorganic particle is selected from a gold particle and an iron oxide particle.

In some embodiments, the metal particle is a magnetic particle. In some embodiments, the inorganic particle is a magnetic particle. In some embodiments, the magnetic particle is a contrast agent for magnetic resonance imaging (MRI). Any magnetic particle suitable for use as an MRI contrast agent may be used in the composition and methods of the present invention. The magnetic particle may be formed, at least in part, from any material affected by a magnetic field. Examples of suitable materials include, but are not limited to magnetite, hematite, ferrites, and materials comprising one or more of iron, cobalt, manganese, nickel, chromium, gadolinium, neodymium, dysprosium, samarium, erbium, iron carbide, iron, or a combination thereof.

In some embodiments, the inorganic particle is a contrast agent for computed tomography (CT) or X-ray imaging. In some embodiments, the inorganic particle is a metal particle which can be used as a CT or X-ray imaging contrast agent. As will be apparent to those skilled in the art, any metal and/or combination of metals suitable for use for imaging by CT or X-ray may be used in the metal particle of the present invention, in embodiments related to diagnostic use. In some embodiments, metals which can be used to form the particle of the invention are heavy metals, or metal with a high Z number. Examples of suitable metals include, but are not limited to: gold, silver, platinum, palladium, cobalt, iron, copper, tin, tantalum, vanadium, molybdenum, tungsten, osmium, iridium, rhenium, hafnium, thallium, lead, bismuth, gadolinium, dysprosium, holmium, and uranium, or a combination thereof.

In some embodiments, the multifunctional particle consists essentially of:
(a) an inorganic particle bound to: (i) a first linear polymeric linker; (ii) a second linear polymeric linker; and (iii) a third linear polymeric linker;
(b) a first immunoglobulin molecule conjugated to the first linear polymeric linker;
(c) a second immunoglobulin molecule conjugated to the second linear polymeric linker; and
(d) a penetration enhancing moiety conjugated to the third linear polymeric linker,
   wherein the length of the third linear polymeric linker is substantially different than the lengths of the first and the second linear polymeric linkers, wherein the first biologically active molecule is distinct from the second biologically active molecule, and wherein the inorganic particle is an imaging agent that can be detected by an imaging modality selected from computed tomography imaging (CT), X-ray imaging, magnetic resonance imaging (MRI), positron emission tomography (PET), single-photon emission computed tomography (SPECT), ultrasound (US), and any combination thereof. Advantageously, in such embodiments, the multifunctional particle can be used in diagnostic applications without the need to conjugate a labeling moiety.

According to some embodiments, the inorganic particle is a metal particle selected from the group consisting of a gold particle, a silver particle, a platinum particle, an iron particle, a copper particle, and a mixture or combination thereof. Each possibility represents a separate embodiment. In some embodiments, the metal particle is a gold (Au) particle.

In some embodiments, the inorganic particle is a metal oxide particle. In some embodiment, the metal oxide particle is selected from the group consisting of iron oxide (Fe₂O₃ or Fe₃O₄), magnesium oxide, nickel oxide, cobalt oxide, aluminum oxide, zinc oxide, copper oxide and manganese oxide, or any combination thereof. Each possibility represents a separate embodiment of the present invention. In some embodiment, the metal oxide particle comprises iron oxide selected from iron (III) oxide and iron (II,III) oxide. In some embodiments, the metal oxide particle is an iron oxide particle wherein the iron oxide is selected from iron (III) oxide and iron (II,III) oxide.

In some embodiments, the core particle is selected from the group consisting of a lipid particle, a carbon-based particle, a ceramic particle, a polymeric particle, and a liposome.

In some embodiments, the core particle is a radiosensitizer. The term “radiosensitizer” as used herein refers to an agent that makes cells (particularly cancer cells) more sensitive to radiation therapy. Typically, materials having high atomic number, such as gold (Z=79) increase radiation sensitivity. Accordingly, a gold nanoparticle is an example of a core particle which is a radiosensitizer.

According to some embodiments, the core particle is a nanoparticle having a diameter of 1-200 nm, 1-180 nm, 1-160 nm, 1-140 nm, 1-120 nm, 1-100 nm, 1-90 nm, 1-80 nm, 1-70 nm, 1-60 nm, 1-50 nm, 1-40 nm, 2-100 nm, 2-60 nm, 2-50 nm, 2-40 nm, 2-30 nm, 2-20 nm, 2-10 nm, 3-100 nm, 3-60 nm, 3-50 nm, 3-40 nm, 3-30 nm, 3-20 nm, 4-100 nm, 4-60 nm, 4-50 nm, 4-40 nm, 5-200 nm, 6-190 nm, 7-180 nm, 8-170 nm, 10-160 nm, 20-160 nm, 10-150 nm, 10-140 nm, 10-120 nm, 10-110 nm, 10-100 nm, 10-90 nm, 10-80 nm, 12-70 nm, 14-60 nm, 15-50 nm, 15-40 nm, 15-30 nm, 20-30 nm, 15-30 nm, 20-90 nm, 20-80 nm, 20-70 nm, 20-60 m, 20-50 nm, 20-40 nm, 20-30 nm, 30-70 nm, 30-60 nm, 40-60 nm, 10-200 nm, 20-200 nm, 30-200 nm, 40-200 nm, 50-200 nm, 60-200 nm, 70-200 nm, 80-200 nm 90-200 nm, 100-200 nm, 110-190 nm, 120-170 nm, 130-160 nm, 100-160 nm, 80-160 nm, 60-160 nm, 40-160 nm, 20-160 nm, 10-160 nm, 20-150 nm or 30-150 nm. Each possibility represents a separate embodiment of the present invention. According to some embodiments, the core particle is nanoparticle having a diameter of at least 1 nm, at least 2 nm, at least 3 nm, at least 4 nm, at least 5 nm, at least 10 nm, at least 12 nm, at least 15 nm, at least 18 nm, at least 20 nm, at least 25 nm, at least 30 nm, at least 35 nm, at least 40 nm, at least 45 nm, at least 50 nm, at least 60 nm, at least 70 nm, at least 80 nm, at least 90 nm, at least 100 nm, at least 110 nm, at least 120 nm, at least 130 nm, at least 140 nm or at least 150 nm. Each possibility represents a separate embodiment of the present invention. According to some embodiments, the core particle is a nanoparticle having a diameter of at most 5 nm, at most 10 nm, at most 15 nm, at most 20 nm, at most 30 nm, at most 40 nm, at most 50 nm, at most 60 nm, at most 70 nm, at most 80 nm, at most 90 nm, at most 100 nm, at most 120 nm, at most 140 nm, at most 160 nm, at most 180 nm or at most 200 nm. Each possibility represents a separate embodiment of the present invention.

According to some embodiments, the multifunctional particle, i.e., the entire co-delivery system has a diameter of 5-500 nm, 6-400 nm, 8-300 nm, 10-300 nm, 10-200 nm, 10-180 nm, 10-160 nm, 10-150 nm, 10-100 nm, 20-90 nm, 20-80 nm, 20-70 nm, 20-60 nm, 25-100 nm, 25-90 nm, 25-80 nm, 25-70 nm, 25-60 nm, 25-50 nm, 30-60 nm, 40-200 nm, 40-150 nm, 40-120 nm, 40-100 nm, 40-80 nm, 40-60 nm, 50-300 nm, 50-250 nm, 50-200 nm, 50-180 nm, 50-150 nm, 60-200 nm, 70-180 nm, 80-180 nm, 90-170 nm, 100-160 nm, 100-200 nm, 150-200 nm or 150-180 nm. According to some embodiments, the multifunctional particle has a diameter of 2-200 nm, 1-100 nm, 1-150 nm, 1-200 nm, 2-50 nm, 2-100 nm, 2-150 nm, 4-50 nm, 4-100 nm, 4-150 nm, or 4-200 nm. Each possibility represents a separate embodiment of the present invention. According to some embodiments, the multifunctional particle has a diameter of at least 1 nm, at least 2 nm, at least 5 nm, at least 10 nm, at least 15 nm, at least 20 nm, at least 25 nm, at least 30 nm, at least 35 nm, at least 40 nm, at least 45 nm, at least 50 nm, at least 55 nm, at least 60 nm, at least 70 nm, at least 80 nm, at least 90 nm, at least 100 nm, at least 110 nm, at least 120 nm, at least 130 nm, at least 140 nm, at least 150 nm, at least 160 nm, at least 180 nm, or at least 200 nm. Each possibility represents a separate embodiment of the present invention. According to some embodiments, the multifunctional particle has a diameter of at most 5 nm, at most 20 nm, at most 30 nm, at most 40 nm, at most 50 nm, at most 60 nm, at most 70 nm, at most 80 nm, at most 90 nm, at most 100 nm, at most 110 nm, at most 120 nm, at most 130 nm, at most 140 nm, at most 150 nm, at most 180 nm, at most 200 nm, at most 250 nm, at most 300 nm, at most 350 nm, at most 400 nm, at most 450 nm or at most 500 nm. Each possibility represents a separate embodiment of the present invention.

As used herein, the term “diameter” of a particle/nanoparticle can be used interchangeably with the term “size” of a particle/nanoparticle and refers to the largest linear distance between two points on the surface of a described particle/nanoparticle. The term “diameter”, as used herein, encompasses sizes of spherical particles as well as of non-spherical particles, and may refer to the actual size of the particle or to its hydrodynamic diameter that includes contributions from the solvation sphere. Any method known in the art can be used to determine the size of the particle, for example transmission electron microscopy (TEM), scanning electron microscopy (SEM), and dynamic light scattering (DLS). The term “diameter” may refer to a mean diameter of a plurality of particles measured by any of the above-mentioned techniques.

The core particle is coated with a polymeric layer comprising at least three polymers: a first polymeric linker which has a functional end group which is capable of binding a first immunoglobulin, a second polymeric linker which has a functional end group which is capable of binding a second immunoglobulin, and a third polymeric linker conjugated to a penetration enhancing moiety.

The term “coated” as used herein is intended to mean that a layer, e.g., a polymeric layer comprising a plurality of polymer moieties, is chemically attached to the surface of the core particle and thereby at least partly covers said core particle. A “particle coated with a polymeric layer” means that each polymer moiety in the polymeric layer is chemically attached to the particle through a functional end group, e.g., a thiol group, of said polymer moiety. In some embodiments, a thiol group of the polymer is conjugated to gold particles by an Au—S bond. The chemical attachment can be covalent, semi-covalent or non-covalent.

The term “polymer moiety” can be interchangeably used with the term “polymer” and refers to a molecule that contains two or more repeating subunits linked in a linear, branched, hyperbranched, dendritic or cyclic sequence, or any combination thereof. In some embodiments, the term “polymer moiety” refers to a molecule that contains at least 3 repeating subunits linked in a linear, branched, hyperbranched, dendritic or cyclic sequence, or any combination thereof.

Examples of subunits include alkylene, arylene, heteroalkylene, amino acid, nucleic acid, saccharide, and the like. Examples of polymer moieties include but are not limited to poly(ethylene glycol) groups, poly(ethylene amine) groups, and poly(amino acid) groups. The terms “polymer moiety” and “polymer” encompass also polymeric linkers. As used herein, the term “polymeric linker” refers to a polymer moiety, which originally comprises at least one functional/reactive group that enables binding to a substance, e.g., a particle. In some embodiments, polymeric linker is a bifunctional polymer having at least two functional/reactive groups that enable binding to at least two substances thereby linking between said at least two substances. In some embodiments, polymeric linker is a monofunctional polymer having one functional/reactive group that enables binding to one substance, e.g., a core particle. It should be understood that the terms “monofunctional”, “bifunctional”, “functional group”, etc., as used herein, relate to the polymeric linker according to its original form prior to attachment to the core particle and/or to the transporter/penetration moiety or to the respective active agent.

In some embodiments, the core particle is bound to a first polymeric linker. In some embodiments, the core particle is bound to a second polymeric linker. In some embodiments, the core particle is bound to a third polymeric linker. In some embodiments, the core particle is bound to a first, a second and a third polymeric linkers.

The term “bound” can be interchangeably used with the term “conjugated”. In some embodiments, bound is covalently conjugated. The terms “covalent attachment”, “covalently attached”, “covalently linked” and “covalently bonded” are used herein interchangeably and refer to the forming of a chemical bonding that is characterized by the sharing of pairs of electrons between atoms. For example, a covalently attached agent coating refers to an agent coating that forms chemical bonds with a functionalized surface of a substrate, as compared to attachment to the surface via other means, for example, adhesion or electrostatic interaction. It will be appreciated that agents (e.g., polymers) attached covalently to a surface can also be bonded via other means in addition to covalent attachment.

In some embodiments, the polymer moieties and/or linkers are attached to the external surface of the core particle via a chemical attachment selected from the group consisting of: covalent attachment, semi-covalent attachment and non-covalent attachment. Each possibility represents a separate embodiment of the present invention. In some embodiments, the polymer moieties and/or linkers are attached to the external surface of the core particle via a semi-covalent attachment. As used herein, the term “semi-covalent attachment” refers to a coordinate bond wherein the shared pair of electrons which form the bond come from the same atom. In the present disclosure, a semi-covalent attachment may occur between a metal particle, e.g., gold particle, and thiol groups.

In some embodiments, at least one of the first, the second and the third polymeric linkers is a linear polymeric linker. In some embodiments, the first polymeric linker is a linear polymeric linker. In some embodiments, the second polymeric linker is a linear polymeric linker. In some embodiments, the third polymeric linker is a linear polymeric linker. In some embodiments, the first and the second polymeric linkers are linear polymeric linkers. In some embodiments, the first and the third polymeric linkers are linear polymeric linkers. In some embodiments, the second and the third polymeric linkers are linear polymeric linkers. In some embodiments, the first, the second and the third polymeric linkers are linear polymeric linkers. In some embodiments, the linear polymeric linker is a bifunctional linear polymer having two functional/reactive groups on the two ends of said linear polymer. In some embodiments, each one of the first, the second and the third polymeric linkers is independently a linear bifunctional polymeric linker having two functional/reactive groups on the two ends of said linear polymer.

As used herein, the term “linear” polymer/polymeric linker refers, in some embodiments, to a polymer/polymeric linker in which at least 80% of monomer units are connected in a linear fashion, i.e., in the form of a single-strand polymer chain. In further embodiments, the term “linear” polymer/polymeric linker refers to a polymer/polymeric linker in which at least 90% of monomer units are connected in a linear fashion. In yet further embodiments, the term “linear” polymer/polymeric linker refers to a polymer/polymeric linker in which about 100% of monomer units are connected in a linear fashion. The term “single-strand polymer chain” as used herein, refers to a polymer chain that comprises monomers connected in such a way that monomer units are joined to each other through two atoms, one on each monomer unit.

In some embodiments, the multifunctional system further comprises an additional polymer moiety bound to the core particle. In some embodiments, the additional polymer moiety is a linear polymer. In some embodiments, the additional polymer moiety is a monofunctional polymer. In some embodiments, the additional polymer moiety is a monofunctional polymeric linker. The additional polymer moiety is thus, in some embodiments, a fourth polymeric linker bound to the core particle. In some embodiments, the fourth linker is used as a capping moiety. In some embodiments, the core particle is bound to a first, second, third and fourth polymeric linkers. In some embodiments, the fourth polymeric linker is monofunctional, i.e., originally having a single functional end group configured for conjugating said polymeric linker to the core particle. In some embodiments, the fourth polymeric linker is a linear monofunctional polymer.

In some embodiments, the first polymeric linker comprises a polymer selected from the group consisting of, but not limited to a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives, and combinations thereof. Each possibility represents a separate embodiment of the invention.

The term “derivative” as used herein refers to a compound whose core structure is the same as, or closely resembles that of, a parent compound, but which has a chemical or physical modification, such as a different or additional group, such as, but not limited to, an alkoxy group, a carboxy group, an amine group, a methoxy group and a thiol group.

In some embodiments, the first polymeric linker comprises a polyether. In some embodiments, the first polymeric linker is a polyether. In some embodiments, the polyether is polyethylene glycol (PEG) or a derivative thereof.

Where appropriate, the abbreviation (PEG) is used in combination with a numeric suffix which indicates the average molecular weight of the PEG. A form of PEG or a PEG species is a PEG or PEG derivative with a specified average molecular weight.

As used herein “PEG or derivatives thereof” refers to any compound including at least one polyethylene glycol moiety. PEGs exist in linear forms and branched forms comprising a multi-arm and/or grafted polyethylene glycols. The term “PEG derivative”, as used herein, relates to PEG which is modified by alkylation of the terminal hydroxy group. In some embodiments, the terminal hydroxyl group is alkylated by a linear or branched C1-C6 alkyl. A PEG may further comprise a functional group. A PEG may be mono-, di-, or multifunctional polyethylene glycols.

Exemplary functional groups include, but are not limited to, the following: a hydroxyl, a carboxyl, a thiol, an amine, a phosphate, a phosphonate, a sulfate, a sulfite, a sulfonate, a sulfoxide, a sulfone, an amide, an ester, a ketone, an aldehyde, a cyano, an alkyne, an azide, and an alkene, or a combination thereof.

In some embodiments, the first polymeric linker comprises a thiol (—SH) end group. In some embodiments, said first polymeric linker is chemically attached to the core particle through said thiol (—SH) end group. In some embodiments, the first polymeric linker is conjugated to the first immunoglobulin through an amide bond. In some embodiments, the core particle is bound to the first polymeric linker through a sulfide bond and the first immunoglobulin is conjugated to said first polymeric linker through an amide bond. In some embodiments, the core particle is an inorganic particle and is bound to the first polymeric linker through a sulfide bond and the first immunoglobulin is conjugated to said first polymeric linker through an amide bond. In some embodiments, the first polymeric linker within the co-delivery system has a structure —S—R—CONH—, wherein R is a polymeric chain consisting of repeating monomer units. In other embodiments, the first polymeric linker within the co-delivery system has a structure —S—R—NHCO—, wherein R is a polymeric chain consisting of repeating monomer units. In some embodiments, the first polymeric linker is selected from thiolated PEG acid (HS-PEG-COOH) and thiolated PEG amine (HS-PEG-NH₂). It is to be understood that the HS and COOH/NH₂ end groups refer to the polymeric linker prior to conjugation with the core particle and the immunoglobulin. In some embodiments, the thiol group is chemically attached to the core particle and the acid or amine group is covalently conjugated to the first immunoglobulin. In some embodiments, the first polymeric linker within the co-delivery system has a structure selected from —S-PEG-C(O)— and —S-PEG-NH—.

In some embodiments, the first polymeric linker is a non-cleavable linker. In some embodiments, the first polymeric linker is non-cleavable under physiological conditions.

The term “non-cleavable” as used herein refers to a stable bond that is not acid or base sensitive, not sensitive to reducing or oxidizing agents, and not sensitive to enzymes that can be found in cells or the circulatory system. In some embodiments, non-cleavable polymeric linkers are devoid of pH sensitive hydrazones. In some embodiments, non-cleavable polymeric linkers are devoid of disulfide bonds. In some embodiments, non-cleavable polymeric linkers are devoid of ester bonds. It is to be understood that the term “polymeric linker is non-cleavable”, is meant to encompass the bond between the core particle and the polymeric linker; the bond between the respective polymeric linker and the respective active agent; or the bond between the respective polymeric linker and the penetration enhancing moiety, as well as any bond within the polymeric linker itself.

In some embodiments, the first polymeric linker has a molecular weight (MW) between 2,000 to 7,000 Da. In some embodiments, the first polymeric linker has a molecular weight (MW) within a range selected from the group consisting of 500-10,000 Da, 1,000-10,000 Da, 600-9,500 Da, 700-9,000 Da, 800-8,500 Da, 800-6,000 Da, 800-5,000 Da, 800-4,000 Da, 800-3,000 Da, 800-2,000 Da, 900-8,000 Da, 1,000-7,000 Da, 1,500-6,500 Da, 2,000-6,000 Da, 3,000-6,000 Da, 4,000-6,000 Da, 1,000-2,000 Da, 1,000-3,000 Da, 1,000-4,000 Da, 1,000-5,000 Da, 1,000-7,000 Da, 3,400-7,000 Da, 2,000-3,000 Da, 2,000-5,000 Da, 2,000-7,000 Da, 2,000-10,000 Da, 3,000-3,400 Da, 3,000-4,000 Da 3,000-5,000 Da, 3,000-7,000 Da, 3,000-10,000 Da, 5,000-7,000 Da, 5,000-10,000 Da, and 7,000-10,000 Da. Each possibility represents a separate embodiment. According to some embodiments, the first polymeric linker has a MW of at least 1,000 Da, at least 1,500 Da, at least 2,000 Da, at least 2,500 Da, at least 3,000 Da, at least 3,400 Da, at least 4,000 Da, at least 5,000 Da, at least 6,000 Da, at least 7,000 Da, or at least 8,000 Da. Each possibility represents a separate embodiment. According to some embodiments, the first polymeric linker has a MW of at most 2,000 Da, at most 3,000 Da, at most 4,000 Da, at most 5,000 Da, at most 6,000 Da, at most 7,000 Da, or at most 10,000 Da. Each possibility represents a separate embodiment.

In some embodiments, the second polymeric linker comprises a polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof. Each possibility represents a separate embodiment of the invention.

In some embodiments, the second polymeric linker comprises a polyether. In some embodiments, the second polymeric linker is a polyether. In some embodiments, the polyether is polyethylene glycol (PEG) or a derivative thereof.

In some embodiments, the second polymeric linker comprises a thiol (—SH) end group. In some embodiments, said second polymeric linker is chemically attached to the core particle through the thiol (—SH) end group. In some embodiments, the second polymeric linker is conjugated to the second immunoglobulin through an amide bond. In some embodiments, the core particle is bound to the second polymeric linker through a sulfide bond and the second immunoglobulin is conjugated to said second polymeric linker through an amide bond. In some embodiments, the core particle is an inorganic particle and is bound to the second polymeric linker through a sulfide bond and the second immunoglobulin is conjugated to said second polymeric linker through an amide bond. In some embodiments, the second polymeric linker within the co-delivery system has a structure —S—R—CONH—, wherein R is a polymeric chain consisting of repeating monomer units. In other embodiments, the second polymeric linker within the co-delivery system has a structure —S—R—NHCO—, wherein R is a polymeric chain consisting of repeating monomer units.

In some embodiments, the second polymeric linker is selected from thiolated PEG acid (HS-PEG-COOH) and thiolated PEG amine (HS-PEG-NH₂). It is to be understood that the HS and COOH/NH₂ end groups refer to the polymeric linker prior to conjugation with the core particle and the immunoglobulin. In some embodiments, the thiol group is chemically attached to the core particle and the acid or amine group is covalently conjugated to the second immunoglobulin. In some embodiments, the second polymeric linker within the co-delivery system has a structure selected from —S-PEG-C(O)— and —S-PEG-NH—.

In some embodiments, the second polymeric linker is a non-cleavable linker. In some embodiments, the second polymeric linker is non-cleavable under physiological conditions.

In some embodiments, the second polymeric linker has a molecular weight (MW) between 2,000 to 7,000 Da. In some embodiments, the second polymeric linker has an MW within a range selected from the group consisting of 500-10,000 Da, 600-9,500 Da, 700-9,000 Da, 800-8,500 Da, 800-6,000 Da, 800-5,000 Da, 800-4,000 Da, 800-3,000 Da, 800-2,000 Da, 900-8,000 Da, 1,000-7,000 Da, 1,500-6,500 Da, 2,000-6,000 Da, 3,000-6,000 Da, 4,000-6,000 Da, 1,000-2,000 Da, 1,000-3,000 Da, 1,000-4000 Da, 1,000-5,000 Da, 1,000-7,000 Da, 1,000-10,000 Da, 2,000-3,000 Da, 2,000-5,000 Da, 2,000-7,000 Da, 2,000-10,000 Da, 3,000-10,000 Da, 3,000-7,000 Da, 3,000-5,000 Da, 3,000-3,400 Da, 3,400-7,000 Da, 5,000-7,000 Da, 5,000-10,000 Da, and 7,000-10,000 Da. Each possibility represents a separate embodiment. According to some embodiments, the second polymeric linker has an MW of at least 1,000 Da, at least 1,500 Da, at least 2,000 Da, at least 2,500 Da, at least 3,000 Da, at least 3,400 Da, at least 4,000 Da, at least 5,000 Da, at least 6,000 Da, at least 7,000 Da or at least 8,000 Da. Each possibility represents a separate embodiment. According to some embodiments, the second polymeric linker has an MW of at most 2,000 Da, at most 3,000 Da, at most 4,000 Da, at most 5,000 Da, at most 6,000, at most 7,000 Da or at most 10,000 Da. Each possibility represents a separate embodiment.

According to some embodiments, the first polymeric linker and the second polymeric linker comprise different polymers. According to some embodiments, the first polymeric linker and the second polymeric linker are different polymers. In some embodiments the first polymeric linker and the second polymeric linker comprise the same polymer. In some embodiments the first polymeric linker and the second polymeric linker are identical.

In some embodiments, the first and second polymeric linkers comprise the same polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof. In some embodiments, both the first and second polymeric linkers comprise PEG. In some embodiments, both the first and second polymeric linkers are PEG. In some embodiments, both the first and second polymeric linkers comprise thiolated PEG. In some embodiments, the first and second polymeric linkers comprise thiolated PEG acid (HS-PEG-COOH) or thiolated PEG amine (HS-PEG-NH₂). In some embodiments, the first and second polymeric linkers are thiolated PEG acid (HS-PEG-COOH) or thiolated PEG amine (HS-PEG-NH₂). In some embodiments, the first and second polymeric linkers are both thiolated PEG acid (HS-PEG-COOH). In some embodiments, the first and second polymeric linkers are both thiolated PEG amine (HS-PEG-NH₂).

In some embodiments, the third polymeric linker comprises a polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof. Each possibility represents a separate embodiment of the invention.

In some embodiments, the third polymeric linker comprises a polyether. In some embodiments, the third polymeric linker is a polyether. In some embodiments, the polyether is polyethylene glycol (PEG) or a derivative thereof.

In some embodiments, the third polymeric linker comprises a thiol (—SH) end group. In some embodiments, said third polymeric linker is chemically attached to the core particle through the thiol (—SH) end group. In some embodiments, the third polymeric linker is conjugated to the penetration moiety, through an amide bond. In some embodiments, the core particle is bound to the third polymeric linker through a sulfide bond and the penetration moiety is conjugated to said third polymeric linker through an amide bond. In some embodiments, the core particle an inorganic particle and is bound to the third polymeric linker through a sulfide bond and a penetration enhancing moiety is conjugated to said third polymeric linker through an amide bond.

In some embodiments, the third polymeric linker within the co-delivery system has a structure —S—R—CONH—, wherein R is a polymeric chain consisting of repeating monomer units. In other embodiments, the third polymeric linker within the co-delivery system has a structure —S—R—NHCO—, wherein R is a polymeric chain consisting of repeating monomer units. In some embodiments, the third polymeric linker is selected from thiolated PEG acid (HS-PEG-COOH) and thiolated PEG amine (HS-PEG-NH₂). It is to be understood that the HS and COOH/NH₂ end groups refer to the polymeric linker prior to conjugation with the core particle and the penetration enhancing moiety. In some embodiments, the thiol group is chemically attached to the core particle and the acid or amine group is covalently conjugated to the penetration enhancing moiety. In some embodiments, the third polymeric linker within the co-delivery system has a structure selected from —S-PEG-C(O)— and —S-PEG-NH—.

In some embodiments, the third polymeric linker has a molecular weight (MW) between 2,000 to 7,000 Da. In some embodiments, the third polymeric linker has an MW within a range selected from the group consisting of 500-10,000 Da, 600-9,500 Da, 700-9,000 Da, 800-8,500 Da, 800-6,000 Da, 800-5,000 Da, 800-4,000 Da, 800-3,000 Da, 800-2,000 Da, 900-8,000 Da, 1,000-7,000 Da, 1,500-6,500 Da, 2,000-6,000 Da, 3,000-6,000 Da, 4,000-6,000 Da, 1,000-2,000 Da, 1,000-3,000 Da, 1,000-4,000 Da, 1,000-5,000 Da, 1,000-7,000 Da, 1,000-10,000 Da, 2,000-3,000 Da, 2,000-5,000 Da, 2,000-7,000 Da, 2,000-10,000 Da, 3,000-10,000 Da, 3,000-7,000 Da, 3,000-5,000 Da, 3,000-3,400 Da, 3,400-7,000 Da, 5,000-7,000 Da, 5,000-10,000 Da, and 7,000-10,000 Da. Each possibility represents a separate embodiment. According to some embodiments, the third polymeric linker has an MW of at least 1,000 Da, at least 1,500 Da, at least 2,000 Da, at least 2,500 Da, at least 3,000 Da, at least 3,400 Da, at least 4,000 Da, at least 5,000 Da, at least 6,000 Da, at least 7,000 Da or at least 8,000 Da. Each possibility represents a separate embodiment. According to some embodiments, the third polymeric linker has an MW of at most 2,000 Da, at most 3,000 Da, at most 4,000 Da, at most 5,000 Da, at most 6,000, at most 7,000 Da or at most 10,000 Da. Each possibility represents a separate embodiment.

In some embodiments, the third polymeric linker is a non-cleavable linker. In some embodiments, the third polymeric linker is non-cleavable under physiological conditions.

In some embodiments, at least one of the first, the second and the third polymeric linkers comprises a cleavable linker. In some embodiments, at least one of the first and the second polymeric linkers comprises a cleavable linker. In some embodiments, each one of the first and the second polymeric linkers independently comprises a cleavable linker. According to some embodiments, the cleavable linker comprises a bond susceptible to cleavage by an endogenous molecule, located or expressed in a specific malignancy, body region or tissue, e.g., breast or other non-CNS tissue or location. In some embodiments, the cleavable linker is PEG succinimidyl succinate (PEGSS). According to some embodiments, the endogenous molecule is glutathione. According to some embodiments, the endogenous molecule is selected from the group comprising of proteases, nucleases, hydronium ions, and reducing agents. In some embodiments, the endogenous molecule is selected from neuroserpin and Serpin B. Each possibility represents a separate embodiment.

According to some embodiments, the multifunctional particle further comprises a cleaving molecule inducer. According to some embodiments, the cleaving molecule inducer is selected from the group comprising of N-acetyl-1-cysteine (NAC), glutathione monoester, γ-glutamylcysteine, γ-glutamylcysteine synthetase, glutathione synthetase. Each possibility represents a separate embodiment.

In some embodiments, the endogenous molecule is glutathione and the cleaving molecule inducer is selected from the group comprising of N-acetyl-1-cysteine (NAC), glutathione monoester, γ-glutamylcysteine, γ-glutamylcysteine synthetase, glutathione synthetase. Each possibility represents a separate embodiment.

According to some embodiments, at least one of the first polymeric linker and the second polymeric linker is different than the third polymeric linker. In some embodiments, at least one of the first polymeric linker and the second polymeric linker comprises the same polymer as the third polymeric linker. In some embodiments, the first polymeric linker, the second polymeric linker and the third polymeric linker comprise the same polymer. In further embodiments, the first polymeric linker is composed of repeating monomer units and the third polymeric linker is composed of the same repeating monomer units as the first linear polymeric linker. In some related embodiments, the first linear polymeric linker has a different number of repeating monomer units than the third linear polymeric linker. In some embodiments, the second polymeric linker is composed of repeating monomer units and the third polymeric linker is composed of the same repeating monomer units as the second linear polymeric linker. In some related embodiments, the second linear polymeric linker has a different number of repeating monomer units than the third linear polymeric linker. In some embodiments, the first and the second polymeric linkers are identical and are composed of repeating monomer units, and the third polymeric linker is composed of the same repeating monomer units as the first and the second linear polymeric linkers. In some related embodiments, the first and the second linear polymeric linkers have a different number of repeating monomer units than the third linear polymeric linker.

In some embodiments, the first, the second and the third polymeric linkers comprise the same polymer selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof. In some embodiments, the first, the second and the third polymeric linkers comprise PEG. In some embodiments, the first, the second and the third polymeric linkers are PEG. In some embodiments, the first, the second and the third polymeric linkers comprise thiolated PEG. In some embodiments, the first, the second and the third polymeric linkers comprise thiolated PEG acid (HS-PEG-COOH) or thiolated PEG amine (HS-PEG-NH₂). In some embodiments, the first, the second and the third polymeric linkers are thiolated PEG acid (HS-PEG-COOH) or thiolated PEG amine (HS-PEG-NH₂). In some embodiments, the first, the second and the third polymeric linkers are thiolated PEG acid (HS-PEG-COOH). In some embodiments, the first, the second and the third polymeric linkers are thiolated PEG amine (HS-PEG-NH₂).

In some embodiments, the first immunoglobulin is covalently conjugated to the first polymeric linker through a first functional end group of said linker, the second immunoglobulin is covalently conjugated to the second polymeric linker through a second functional end group of said linker, and the penetration enhancing moiety is covalently conjugated to the third polymeric linker through a third functional end group of said linker. Exemplary functional end group include but not limited to thiol group, carboxylic group, and amine group. In some embodiments, at least two of the first functional end group, the second functional end group and the third functional end group, are identical. In some embodiments, the first functional end group and the second functional end group are identical. In some embodiments, the first functional end group and the third functional end group are identical. In some embodiments, the second functional end group and the third functional end group are identical. In some embodiments, the first functional end group, the second functional end group and the third functional end group are identical.

In some embodiments, the first functional end group and the second functional end group are different. In some embodiments, the first functional end group and the third functional end group are different. In some embodiments, the second functional end group and the third functional end group are different.

In some embodiments, the first, the second and the third polymeric linkers are linear. According to the principles of the present invention, the length of the third polymeric linker is substantially different than the length of at least one of the first and the second polymeric linkers. In some embodiments, the length of the third polymeric linker is substantially different than the length of the first polymeric linker. In some embodiments, the length of the third polymeric linker is substantially different than the length of the second polymeric linker. In some embodiments, the length of the third polymeric linker is substantially different than the length of both the first polymeric linker and the second polymeric linker. In some embodiments, the length of the first polymeric linker is substantially similar to the length of the second polymeric linker and the length of the third polymeric linker is substantially different than the length of both the first polymeric linker and the second polymeric linker.

In some embodiments, the term “length” of a polymeric moiety or linker refers to the length of the polymer which depends on the number of monomers incorporated therein, the length of each monomer unit, the polymer chain structure (for example, whether the polymer is linear or branched), spatial conformation, deformation of valent (or binding angels) angles, and the degree of stretching or coiling.

The length of a polymer can be calculated as known in the art, for example as described in Introduction to Physical Polymer Science, Fourth Edition, L. H. Sperling, First published: 4 Nov. 2005, Chapter 3. Additionally, various computational modeling methods, which can be performed using, inter alia, Hyperchem, ACD/3D, MOE 2010.10, or Chem 3D software. can be used for evaluating the length of a polymer, as known in the art. Physical characterization methods, such as, for example, static light scattering, can also be used to assess the length of a coiled polymer. It is to be understood that when assessing the difference between the length of polymeric linkers, the same length definitions (or length measurement methods) must be used for the compared polymeric linkers.

The term “length” when referring to a linear polymer can refer to different length definitions. According to some embodiments, the term “length” refers to a displacement length, also termed herein “end-to-end” length, which is the distance between two ends of the polymer chain for a coiled polymer. End-to-end length can be expressed, for example, as Flory radius:

$$F = α {n}^{3} {/}_{5} \qquad Equation I$$

wherein F=Flory radius, a=monomer dimension, n=degree of polymerization,

According to some embodiments, the term “length” refers to contour length, which is the distance between two ends of the polymer chain when the polymer is stretched out. The contour length could be considered the maximum possible displacement length. Contour length (also termed herein “old contour length”) can be calculated by dividing MW of the polymer by the MW of the monomer unit and multiplying by the length of the monomer unit. To account for binding angles, the contour length (also termed herein “new contour length”) can be calculated by dividing MW of the polymer by the MW of the monomer unit, multiplying by the length of the monomer unit and further multiplying by cosine of the ((binding angle theta-180)/2).

As explained hereinabove, the length of a linear polymer can be estimated based on its molecular weight and chemical structure of a monomer unit. In order to evaluate the difference between the polymeric linkers which comprise the same polymer (i.e., composed of the same type but a different number of monomer units), molecular weights of the polymeric linkers can conveniently be used. Accordingly, in some embodiments, the molecular weight of the third polymeric linker is substantially different than the molecular weights of at least one of the first and the second linear polymeric linkers. In some embodiments, the molecular weight of the third polymeric linker is substantially different than the molecular weight of the first polymeric linker.

In some embodiments, the molecular weight of the third polymeric linker is substantially different than the molecular weight of the second polymeric linker. In some embodiments, the molecular weight of the third polymeric linker is substantially different than the molecular weights of the first and the second polymeric linker.

As used herein, the term “substantially different” refers to a difference of at least 5%, at least 6%, at least 7%, at least 8%, at least 9%, at least 10%, at least 12%, at least 15%, at least 18%, at least 20%, at least 25%, at least 30%, at least 35%, at least 40%, at least 45%, or at least 50%. Each possibility represents a separate embodiment of the present invention. The term “substantially higher” means that a first value is higher than a second value wherein the difference between the first and the second values is at least 5%, at least 6%, at least 7%, at least 8%, at least 9%, at least 10%, at least 12%, at least 15%, at least 18%, at least 20%, at least 25%, at least 30%, at least 35%, at least 40%, at least 45%, or at least 50%. Each possibility represents a separate embodiment of the present invention.

In some embodiments, the molecular weight of the monomeric unit of the third polymeric linker is substantially similar to the molecular weight of the monomeric unit of the at least one of the first and the second polymeric linkers. As used herein, the term “substantially similar” refers to a similarity of at least 70%, at least 75%, at least 80%, at least 85%, at least 90%, at least 91%, at least 92%, at least 93%, at least 94%, or at least 95%. Each possibility represents a separate embodiment of the present invention.

In some embodiments, the third polymeric linker and at least one of the first polymeric linker and the second polymeric linker comprise the similar polymer. In some embodiments, the third linear polymeric linker is composed of repeating monomer units and at least one of the first polymeric linker and the second linear polymeric linker is composed of the same repeating monomer units as the third linear polymeric linker, wherein the third linear polymeric linker has a different number of repeating monomer units than the at least one of the first polymeric linker and the second linear polymeric linker. In some embodiments, the third polymeric linker and the at least one of the first polymeric linker and the second polymeric linker are similar except for the length of said third and said first and/or second polymeric linkers.

In some embodiments, the third polymeric linker and the at least one of the first polymeric linker and the second polymeric linker, have a difference in their respective molecular weights of at least about 100 Da, at least about 150 Da, at least about 200 Da, at least about 250 Da, at least about 300 Da, at least about 350 Da, at least about 400 Da, at least about 450 Da, at least about 500 Da, at least about 550 Da, at least about 600 Da, at least about 650 Da, at least about 700 Da, at least about 750 Da, at least about 800 Da, at least about 850 Da, at least about 900 Da, at least about 950 Da, at least about 1000 Da, at least about 1100 Da, at least about 1200 Da, at least about 1300 Da, at least about 1400 Da, at least about 1500 Da, at least about 1600 Da, at least about 1700 Da, at least about 1800 Da, at least about 1900 Da, or at least about 2000 Da. Each possibility represents a separate embodiment of the present invention.

In some embodiments, the difference between the lengths of third polymeric linker and the at least one of the first polymeric linker and the second linear polymeric linker, is configured to enable exposure of the penetration enhancing moiety on the external surface of the co-delivery system, which faces the tumor cell membrane or the external surface of the solid tumor. It is to be understood that the immunoglobulins are not enclosed or encapsulated within the core particle, but rather are attached to the external surface thereof, via a polymeric linker, similarly to the internalizing moiety, which is also attached to the surface of the same core particle via a polymeric linker. Without wishing to being bound by theory or mechanism of action, it is contemplated that attaching the penetration enhancing/internalizing moiety through a polymeric chain having similar length as the first and/or second polymeric linkers, may prevent sufficient exposure of said internalizing moiety on the external surface of the co-delivery system, and thereby limit the penetration of the system through cell membranes and through blood-tumor barrier.

Without further wishing to being bound by theory or mechanism of action, it is contemplated that the active immunoglobulins, which are not enclosed or encapsulated within the core particle, remain accessible for effectively binding their antigen despite being bound to the multifunctional system. Advantageously, the specific composition of the multifunctional system of the invention which ensures formation of a conjugated particle with a particular hierarchical structure, not only allows to deliver various types of immunoglobulin combinations, but also does not interfere with the functionality of the immunoglobulins, such that cleavage of the linkage between the active agents and the core particle after penetration through a biological membrane, is not necessarily required.

Accordingly, in some embodiments, the molecular weight of the third polymeric linker is higher than the molecular weight of at least one of the first and the second polymeric linker. In further embodiments, the molecular weight of the third polymeric linker is higher than the molecular weight of both the first and the second polymeric linkers. In some embodiments, the molecular weight of the third polymeric linker is higher than the molecular weight of the first and/or the second polymeric linker provided that the molecular weight of said first and/or the second polymeric linker is less than 4950 Da. linkers. In some embodiments, the molecular weight of the third polymeric linker is higher than the molecular weight of the first and/or the second polymeric linker provided that the molecular weight of said first and/or the second polymeric linker is less than 4900 Da. In some embodiments, the molecular weight of the third polymeric linker is higher than the molecular weight of the first and/or the second polymeric linker provided that the molecular weight of said first and/or the second polymeric linker is less than 4800 Da. In some embodiments, the molecular weight of the third polymeric linker is higher than the molecular weight of the first and/or the second polymeric linker provided that the molecular weight of said first and/or the second polymeric linker is less than 4780 Da. In some embodiments, the third polymeric linker is a PEG derivative having a molecular weight of about 5000 Da and at least one of the first and the second polymeric linkers is a PEG derivative having a molecular weight of about 3500 Da. In some embodiments, the third polymeric linker is a PEG derivative having a molecular weight of about 5000 Da, and the first and the second polymeric linkers are both PEG derivatives having a molecular weight of about 3500 Da.

In some embodiment, the third polymeric linker has a molecular weight which is higher than the molecular weight of at least one of the first and the second polymeric linkers. In some embodiments, the MW of the polymeric linkers directly depend on the relative molecular weights of the immunoglobulin and the penetration enhancing moiety. In some embodiments, the first immunoglobulin molecule has a higher MW than the transport moiety and the first polymeric linker has a lower MW than the third polymeric linker. In some embodiments, the second immunoglobulin molecule has a higher MW than the transport moiety and the second polymeric linker has a lower MW than the third polymeric linker.

In some embodiment, the third polymeric linker is longer than the first and/or the second polymeric linker. In some embodiments, the third polymeric linker has a higher end-to-end distance than the first and/or the second polymeric linker. In some embodiments, the third polymeric linker has a higher contour distance than the first and/or the second polymeric linker.

In some embodiments, the third polymeric linker has a MW smaller than the MW of at least one of the first and the second polymeric linkers. In some related embodiments, the MW of the at least one of the first and the second polymeric linkers is at least about 4000 Da. In further related embodiments, the difference between the Mw of the first polymeric linker and the at least one of the first and the second polymeric linkers is at least about 2000 Da. Without wishing to being bound by theory or mechanism of action, it is contemplated that the significantly longer first and/or second linker allows folding of the polymer chain (or a higher degree of coiling), such that the actual distance between the respective immunoglobulin and the core particle is smaller than between the penetration moiety and the core particle, such that the immunoglobulin is at least partly shielded by the transported moiety which is exposed on the surface of the multifunctional particle during membrane penetration. In some related embodiments, the end-to-end distance of the third polymeric linker is higher than the end-to-end distance of the first and/or the second polymeric linker, despite the higher MW of said first and/or second polymeric linker.

In some embodiments, the distance between the first immunoglobulin and the core particle and the distance between the second immunoglobulin and the core particle, are smaller than the distance between the transport moiety and the core particle. In some embodiments, at least one end group of the third polymeric linker is similar to at least one end group of the first polymeric linker. In some embodiments, at least one end group of the third polymeric linker is similar to at least one end group of the second polymeric linker. In some embodiments, the two end groups of the third polymeric linker are similar to the two end groups of the first polymeric linker. In some embodiments, the two end groups of the third polymeric linker are similar to the two end groups of the second polymeric linker. In some embodiments, the two end groups of the first polymeric linker are similar to the two end groups of the second polymeric linker.

In some embodiments, the core particle is bound to an additional, fourth, polymer. In some embodiments, said polymer is a monofunctional polymeric linker. In some embodiments, the core particle is coated with a polymeric layer comprising the first polymeric linker, the second polymeric linker, the third polymeric linker and additional, fourth, polymeric linker wherein the additional polymeric linker is monofunctional. The terms “fourth polymer” and “fourth polymeric linker” can be used interchangeably. In some embodiments, the fourth polymer functions as a spacer moiety. In some embodiments, the fourth polymeric linker is a linear polymeric linker. In some embodiments, the fourth polymer is selected from the group consisting of a polyether, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, derivatives and combinations thereof.

As used herein, the term “monofunctional” means that the polymer before being conjugated to the core particle has only one functional group configured to bind said polymer to the core particle. The monofunctional polymeric linker is therefore neither conjugated nor capable of conjugating any moiety except for the core particle.

In some embodiments, the fourth polymer comprises the same monomer units as the first and/or the second polymers. In some embodiments, the fourth polymer comprises the same monomer units as the third polymeric linker. In some embodiments, the first, the second, the third and the fourth polymers comprise the same monomer units. In some embodiments, the fourth polymer is bound to the core particle through a thiol end group of said polymer. In some embodiments, the fourth polymer is a polyether. In some embodiments, the polyether is methoxy polyethylene glycol (mPEG) or a derivative thereof. In some embodiments, the mPEG is thiolated (mPEG-SH) wherein said thiolated mPEG is bound to the core particle via the thiol end group.

In some embodiments, the fourth polymer has a MW between 1,000 to 7,000 Da. In some embodiments, the fourth polymer has a MW from 500-1,000 Da, 500-3,000 Da, 500-7,000 Da, 500-10,000 Da, 1,000-3,000 Da, 1,000-4,000 Da, 1,000-5,000 Da, 1,000-7,000 Da, 1,000-10,000 Da, 3,000-5,000 Da, 3,000-7,000 Da, 3,000-10,000 Da, 7. Each possibility represents a separate embodiment. According to some embodiments, the fourth polymer has a MW of at least 1,000 Da, at least 2,000 Da, at least 3,000 Da, at least 4,000 Da, at least 5,000 Da, at least 6,000 Da, at least 7,000 Da, or at least 8,000 Da. Each possibility represents a separate embodiment. According to some embodiments, the fourth polymer has a MW of at most 1,000 Da, at most 2,000 Da, at most 3,000 Da, at most 4,000 Da, at most 5,000 Da, at most 6,000 Da, at most 7,000 Da, or at most 10,000 Da. Each possibility represents a separate embodiment.

In some embodiments, the length of the fourth polymer is substantially similar to the length of at least one of the first polymeric linker, the second polymeric linker and the third polymeric linker. In some embodiments, the length of the fourth polymer is substantially similar to the length of the first polymeric linker. In some embodiments, the length of the fourth polymer is substantially similar to the length of the second polymeric linker. In some embodiments, the length of the fourth polymer is substantially similar to the length of the third polymeric linker. In some embodiments, the length of the fourth polymer is substantially similar to the length of the polymeric linker (first, second or third) that its length is higher than the length of at least one of the other polymeric linkers. In some embodiments, the molecular weight of the fourth polymer is substantially similar to the molecular weight of the polymeric linker (first, second or third) having higher molecular weight than at least one of the other polymeric linkers. In some embodiments, the MW of the fourth polymer is substantially similar to the MW of the first polymeric linker. In some embodiments, the MW of the fourth polymer is substantially similar to the MW of the second polymeric linker. In some embodiments, the MW of the fourth polymer is substantially similar to the MW of the third polymeric linker.

Without wishing to being bound by theory or mechanism of action, the efficacy of the co-delivery system of the invention also depends on molar ratio of the different polymeric linkers, wherein said ratio defines the density of the penetration enhancing moiety and the immunoglobulins of the co-delivery system.

In some embodiments, the first polymeric linker constitutes about 5-70% mol, 5-60% mol, 5-40% mol, 8-60% mol, 10-60% mol, 10-55% mol, 10-50% mol, 10-40% mol, 10-30% mol, 10-25% mol, 10-20% mol, 15-60% mol, 15-55% mol, 15-50% mol, 15-45% mol, 15-40% mol, 15-30% mol, 15-25% mol, 15-20% mol, 2-10% mol, 2-20% mol, 2-50% mol, 2-60% mol, 2-70% mol, 5-10% mol, 5-20% mol, 5-70% mol, 10-20% mol, 10-50% mol, 10-70% mol, 20-50% mol, 20-40% mol, 30-50% mol, 30-60% mol, 30-70% mol, 50-60% mol or 50-70% mol of the total polymers bound to the core particle. Each possibility represents a separate embodiment of the present invention. In some embodiments, first polymeric linker constitutes at least 2% mol, at least 4% mol, at least 5% mol, at least 6% mol, at least 8% mol, at least 10% mol, at least 12% mol, at least 15% mol, at least 18% mol, at least 20% mol, at least 25% mol, at least 30% mol, at least 35% mol, at least 40% mol, at least 50% mol, or at least 60% mol of the total polymers bound to the core particle. Each possibility represents a separate embodiment.

In some embodiments, the second polymeric linker constitutes about 5-70% mol, 5-60% mol, 5-40% mol, 8-60% mol, 10-60% mol, 10-55% mol, 10-50% mol, 10-40% mol, 10-30% mol, 10-25% mol, 10-20% mol, 15-60% mol, 15-55% mol, 15-50% mol, 15-45% mol, 15-40% mol, 15-30% mol, 15-25% mol, 15-20% mol, 2-10% mol, 2-20% mol, 2-50% mol, 2-60% mol, 2-70% mol, 5-10% mol, 5-20% mol, 5-70% mol, 10-20% mol, 10-50% mol, 10-70% mol, 20-50% mol, 20-40% mol, 30-50% mol, 30-60% mol, 30-70% mol, 50-60% mol or 50-70% mol of the total polymers bound to the core particle. Each possibility represents a separate embodiment of the present invention. In some embodiments, the second polymeric linker constitutes at least 2% mol, at least 4% mol, at least 5% mol, at least 6% mol, at least 8% mol, at least 10% mol, at least 12% mol, at least 15% mol, at least 18% mol, at least 20% mol, at least 25% mol, at least 30% mol, at least 35% mol, at least 40% mol, at least 50% mol, or at least 60% mol of the total polymers bound to the core particle. Each possibility represents a separate embodiment.

In some embodiments, the third polymeric linker constitutes about 5-70% mol, 5-60% mol, 5-40% mol, 8-60% mol, 10-60% mol, 10-55% mol, 10-50% mol, 10-40% mol, 10-30% mol, 10-25% mol, 10-20% mol, 15-60% mol, 15-55% mol, 15-50% mol, 15-45% mol, 15-40% mol, 15-30% mol, 15-25% mol, 15-20% mol, 2-10% mol, 2-20% mol, 2-50% mol, 2-60% mol, 2-70% mol, 5-10% mol, 5-20% mol, 5-70% mol, 10-20% mol, 10-50% mol, 10-70% mol, 20-50% mol, 20-40% mol, 30-50% mol, 30-60% mol, 30-70% mol, 50-60% mol or 50-70% mol of the total polymers bound to the core particle. Each possibility represents a separate embodiment of the present invention. In some embodiments, the third polymeric linker constitutes at least 2% mol, at least 4% mol, at least 5% mol, at least 6% mol, at least 8% mol, at least 10% mol, at least 12% mol, at least 15% mol, at least 18% mol, at least 20% mol, at least 25% mol, at least 30% mol, at least 35% mol, at least 40% mol, at least 50% mol, or at least 60% mol of the total polymers bound to the core particle. Each possibility represents a separate embodiment.

In some embodiments, the fourth polymer constitutes about 5-90% mol, 5-85% mol, 5-80% mol, 10-80% mol, 20-78% mol, 25-75% mol, 30-75% mol, 40-75% mol, 50-75% mol, 60-75% mol, 60-70% mol, 60-80% mol, 5-60% mol, 10-60% mol, 10-55% mol, 10-50% mol, 10-40% mol, 15-60% mol, 15-55% mol, 15-50% mol, 15-45% mol or 15-40% mol of the total polymers bound to the core particle. Each possibility represents a separate embodiment of the present invention. In some embodiments, the fourth polymer constitutes between 60-80% mol of the total polymers bound to the core particle. In some embodiments, the fourth polymer constitutes between 50-80% mol of the total polymers bound to the core particle. In some embodiments, the fourth polymer constitutes at least 2% mol, at least 4% mol, at least 5% mol, at least 6% mol, at least 8% mol, at least 10% mol, at least 12% mol, at least 15% mol, at least 18% mol, at least 20% mol, at least 25% mol, at least 30% mol, at least 35% mol, at least 40% mol, at least 45% mol, at least 50% mol, at least 55% mol, at least 60% mol, at least 65% mol, or at least 70% mol of the total polymers bound to the core particle. Each possibility represents a separate embodiment. In some embodiments, the first polymeric linker constitutes about 5 to 45% mol, the second polymeric linker constitutes about 5 to 45% mol, the third polymeric linker constitutes about 10 to 45% mol, and the fourth polymer constitutes about 40 to 80% mol of the total polymers bound to the core particle.

In some embodiments, the first polymeric linker constitutes about 200 to 2000 molecules per particle, the second polymeric linker constitutes about 200 to 2000 molecules per particle, the third polymeric linker constitutes about 400 to 2000 molecules per particle, and the fourth polymer constitutes about 1800 to 3000 molecules per particle.

If was found that particles containing about 2 to 40 antibody molecules per each particle, are capable of binding their cellular targets and eliciting the desired effect. The delivery system of the present invention comprises according to some embodiments, particles carrying about 2 to 50 antibody molecules. According to some embodiments, about 4 to 40 antibody molecules are conjugated, through a linker, to each particle. According to yet other embodiments, about 5 to 30 antibody molecules are conjugated, through a linker, to each particle. According to some specific embodiments, about 2 to 20, 2 to 10, 5 to 15, 10 to 20 or about 15 to 25 antibody molecules are conjugated, through a linker, to each particle.

Yet, when antibody fragments are conjugated to the particle, their respecting number is higher. The delivery system of the present invention thus comprises according to some embodiments, particles carrying about 20 to 400 antibody fragments. According to some embodiments, about 40 to 400 antibody fragments are conjugated, through a linker, to each particle. According to yet other embodiments, about 50 to 300 antibody fragments are conjugated, through a linker, to each particle. According to some specific embodiments, about 20 to 200, 20 to 100, 100 to 200 or about 150-250 antibody fragments are conjugated, through a linker, to each particle.

According to some embodiments, the total amount of the immunoglobulin molecules bound to the nanoparticle does not exceed about 0.2% of the total particle surface capacity. According to some embodiments, the amount of the transporter does not exceed about 0.5% of the particle surface capacity. According to some embodiments, the amount of the transporter does not exceed about 1% of the particle surface capacity.

According to some embodiments, the fourth linker is conjugated to about 90% to 99% of a total surface capacity of the particle.

According to some embodiments, about 20 to 500 antibody fragments are conjugated to each particle, through a linker. According to yet other embodiments, about 40 to 400 antibody fragments are conjugated to each particle, through a linker. According to yet other embodiments, about 50 to 300 antibody fragments are conjugated to each particle, through a linker. According to some specific embodiments, about 20 to 200, 20 to 100, 100 to 200 or about 50-350 antibody fragments are conjugated to each particle through a linker.

In some embodiments, the first polymeric linker constitutes about 10 to 40% mol, the second polymeric linker constitutes about 10 to 40% mol, the third polymeric linker constitutes about 10 to 40% mol, and the fourth polymer constitutes about 40 to 70% mol of the total polymers bound to the core particle.

In some embodiments, the first and second polymeric linkers together constitute about 10% to 60% mol, 10 to 50% mol, 10 to 45% mol, 10 to 40% mol, 10 to 30% mol or 10 to 20% mol of the total polymeric linkers bound to the core particle. Each possibility represents a separate embodiment of the present invention.

It is to be understood that the % mol of each polymer is dependent on the other polymers bound to the core particle, such that the total % mol of the polymers does not exceed 100%.

In some embodiments, the first polymeric linker, second polymeric linker, third polymeric linker and fourth polymer are in a (w/w/w/w) ratio of between 2.5:2.5:5:85 to 20:20:30:30 of the total polymeric linkers bound to the core particle. According to some specific embodiments, the first polymeric linker, second polymeric linker, third polymeric linker and fourth polymer are in a (w/w/w/w) ratio of 2.5:2.5:15:80. According to other embodiments, the first polymeric linker, second polymeric linker, third polymeric linker and fourth polymer are in a (w/w/w/w) ratio of 5:5:10:80 or in a (w/w/w/w) ratio of 10:10:20:60. Each possibility represents a separate embodiment of the present invention.

According to the present invention, the co-delivery system comprises a penetration enhancing moiety conjugated to the third polymeric linker. The terms “penetration enhancing moiety”, “transporter”, ‘penetration enhancer”, “permeability enhancer”, “permeability enhancing”, “internalization transporter” and “internalization enhancer” are used herein interchangeably and refer to moieties that are capable of facilitating or enhancing penetration or internalization of the delivery system through biological membranes, e.g., the blood-tumor barrier, or enables adherence of the delivery system to cancerous cells through a specific receptor, e.g., an insulin receptor. In some embodiments, the transporter also enhances glucose absorption by tumor cells and/or enhances tumor cell metabolism.

In some embodiments, the penetration enhancing moiety is selected from, but not limited to, insulin, an antibody specific for an insulin receptor, or part of such antibody-such as Fab fragment, a polypeptide that specifically binds to the insulin receptor, insulin-like growth factor 1, an antibody specific for an insulin-like growth factor receptor 1, or part of such antibody, a polypeptide that specifically binds to the insulin-like growth factor receptor 1, a cell-penetrating peptide (CPP), and a glucose or a glucose derivative. Each possibility represents a separate embodiment of the present invention.

A “cell-penetrating peptide” (CPP), is a peptide that has an enhanced ability to cross cell membrane bilayer without causing a significant lethal membrane damage.

Other cellular proteins capable of facilitating endocytosis that are known in the art can also be used as penetration enhancing moieties. In some embodiments, the penetration enhancing moiety is a moiety that facilitates or enhances glucose absorption or consumption by tumor cells. In some embodiments, the penetration enhancing moiety is selected form insulin, insulin derivative, glucose or glucose derivative. According to some embodiments, the penetration enhancer is 2-deoxy-D-glucose.

In some embodiments, the MW of the penetration enhancing moiety is about 150 to about 8000 Dalton. In some embodiments, the MW of the penetration enhancer is about 2 kD to about 8 kD. In some embodiments, the MW of the penetration enhancer is about 5 kD.

According to the principles of the present invention, the first polymeric linker is conjugated to a first immunoglobulin and the second polymeric linker is conjugated to a second, distinct immunoglobulin. As used herein, the term “immunoglobulin” refers to an agent that is intended to be delivered into a cell, tissue or tumor of a subject, located outside the brain or outside the CNS, and is capable of being used as a therapeutic, targeting or diagnostic agent. In some embodiments, each one of the first immunoglobulin and the second immunoglobulin is independently selected from a biologically active molecule and a labeling molecule. According to some embodiments, the immunoglobulin molecules are characterized by a poor tissue permeability

In some embodiments, the immunoglobulin molecule is contiguous to the respective polymeric linker. The terms “immunoglobulin” and “immunoglobulin molecule” are used herein interchangeably and refer to compounds or molecules that are capable of binding to specific cellular receptors/antigens/markers and thereby targeting the system to specific cells. In some embodiments, the immunoglobulin molecule is a therapeutic antibody or antibody fragment. In some embodiments, the immunoglobulin molecule has therapeutic applications. In some embodiments, the immunoglobulin molecule has diagnostic applications. In some embodiments, the immunoglobulin molecule has both therapeutic and diagnostic applications. In some embodiments, the immunoglobulin molecule is an intact antibody, a scFv or an antibody fragment. Each possibility represents a separate embodiment of the present invention.

In some embodiments, the first immunoglobulin and/or the second immunoglobulin is a monoclonal antibody. In some specific embodiments, the antibody is selected from the group the group consisting of anti-IgG1, anti-IbA1, anti-HER2+(Trastuzumab & Pertuzumab), anti-EGFR (Cetuximab), anti-GD2 and checkpoint inhibitor antibodies such as anti PD-1, anti PD-L1 and anti-CTLA-4, or a fragment thereof.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site.

An antibody may be oligoclonal, polyclonal, monoclonal, chimeric, camelid, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody), nanobody, and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH) Fc fusions and scFv-scFv-Fc fusions.

The term “antibody” is used in the broadest sense and includes monoclonal antibodies (including full length or intact monoclonal antibodies), polyclonal antibodies, multivalent antibodies, chimeric antibodies, humanized antibodies, and antibody fragments long enough to exhibit the desired biological activity, namely binding to the target of interest.

The mAbs of the present invention may be of any immunoglobulin class including IgG, IgM, IgE, IgA, and IgD.

Chimeric, humanized, and human antibodies comprise, according to some embodiments, a human constant region selected from the group consisting of: IgG1, IgG2, IgG3, and IgG4.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. According to some embodiments, some FR residues in a humanized antibody are substituted with corresponding residues from a non-human antibody (e.g., the antibody from which the CDR residues are derived), e.g., to restore or improve antibody specificity or affinity.

A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

In some embodiments, the first immunoglobulin and/or the second immunoglobulin is an antibody. In some embodiments, the antibody is an antibody that binds specifically to a receptor present on the surface of target cells, e.g., tumor cells or immune cells in the body. In some embodiments, the antibody is an antibody that binds specifically to a receptor present on cells in a specific tissue or body region, e.g., cancerous area or cells. In some embodiments, the antibody is an antibody that binds specifically to a receptor present on the surface of diseased or cancerous cells located outside the brain. In some embodiments, the antibody is a bi-specific antibody. In some embodiments, the first and the second immunoglobulin molecules are both bi-specific antibodies. In some embodiments, the first immunoglobulin and/or the second immunoglobulin is an antibody having a therapeutic activity against cancer.

Exemplary antibodies include but are not limited to: anti-HER2+(Trastuzumab & Pertuzumab), anti-EGFR (Cetuximab), checkpoint inhibitor antibodies (anti PD-1, Anti PD-L1, Anti-CTLA-4), and anti-GD2.

According to some embodiments, at least one antibody in the delivery system is against a target selected from the group consisting of: 1-40-β-amyloid, 4-1BB (CD137), 5AC, activated F9, F10, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta amyloid, C5, CA-125, CA-125 (imitation), calcitonin, Canis lupus familiaris IL31, carbonic anhydrase 9 (CA-IX), cardiac myosin, CCL11 (eotaxin-1), CCR2, CCR4, CCR5, CD11, CD18, CD125, CD140a, CD147 (basigin), CD15, CD152, CD154 (CD40L), CD19, CD2, CD20, CD200, CD22, CD23 (IgE receptor), CD25 (a chain of IL-2 receptor), CD27, CD4, CD6, CD28, CD3, CD3 epsilon, CD30 (TNFRSF8), CD33, CD37, CD38, CD4, CD40, CD44 v6, CD5, CD51, CD52, CD56, CD6, CD70, CD74, CD79B, CD80, CEA, CEA-related antigen, CFD, CGRP, ch4D5, CLDN18.2, Clostridium difficile, clumping factor A, coagulation factor III, CSFIR, CSF2, CTGF, CTLA-4, CXCR4 (CD184), cytomegalovirus, cytomegalovirus glycoprotein B, dabigatran, DLL3, DLL4, DPP4, DR5, E. coli shiga toxin type-1, E. coli shiga toxin type-2, EGFL7, EGFR, endoglin, endotoxin, EpCAM, ephrin receptor A3, episialin, ERBB3 (HER3), F protein of respiratory syncytial virus, FAP, FGF 23, fibronectin extra domain-B, folate hydrolase, folate receptor alpha, Frizzled receptor, GCGR, GD2 ganglioside, GDF-8, glypican 3, GMCSF receptor α-chain, GPNMB, growth differentiation factor 8, GUCY2C, hemagglutinin, hepatitis B surface antigen, HER1, HER2/neu, HGF, HHGFR, histone complex, HIV-1, HLA-DR, HNGF, human beta-amyloid, human TNF, ICAM-1 (CD54), ICOSL, IFN-α, IFN-γ, IgE, IgE Fc region, IGF-1 receptor (CD221), IGF1, IGF2, IGHE, IL 17A, IL 17A and IL 17F, IL 20, IL-1, IL-12, IL-23, IL-13, IL-17, IL17A and IL17F, ILIA, IL-1β, IL2, IL-22, IL23, IL23A, IL31RA, IL-4, IL-5, IL-6, IL-6 receptor, IL9, ILGF2, influenza A virus hemagglutinin HA, integrin α4, integrin α4β7, integrin α5β1, integrin α7β7, integrin αvβ3, interferon gamma-induced protein, interferon receptor, interferon α/β receptor, ITGA2 (CD49b), kallikrein, KIR2D, KLRC1, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch 1, Notch receptor, NRP1, Oryctolagus cuniculus, OX-40, oxLDL, PCSK9, PD-1, PDCDI, PDGF-R a, phosphate-sodium co-transporter, phosphatidylserine, platelet-derived growth factor receptor beta, prostatic carcinoma cells, Pseudomonas aeruginosa, Pseudomonas aeruginosa type III secretion system, rabies virus glycoprotein, RANKL, respiratory syncytial virus, RHD, Rhesus factor, RON, RTN4, scatter factor receptor kinase, sclerostin, SDC1, selectin P, SLAMF7, SOST, sphingosine-1-phosphate, Staphylococcus aureus, STEAPI, TAG-72, TEM1, tenascin C, TFPI, TGF beta 1, TGF beta 2, TGF-β, TNFR superfamily member 4, TNF-α, TRAIL-R1, TRAIL-R2, TSLP, tumor antigen CTAA16.88, tumor specific glycosylation of MUC1, tumor-associated calcium signal transducer 2, TWEAK receptor, TYRP1 (glycoprotein 75), VEGF, VEGF-A, VEGFR-1, VEGFR2, vimentin, VWF, and any combination thereof.

According to some embodiments, the antibody is selected from the group consisting of Abagovomab, Abituzumab, Abrilumab, Actoxumab, Adalimumab, Adecatumumab, Aducanumab, Afasevikumab, Afutuzumab, ALD518, Alemtuzumab, Alirocumab, Altumomab pentetate, Amatuximab, Anetumab ravtansine, Anifrolumab, Anrukinzumab (IMA-638), Apolizumab, Ascrinvacumab, Aselizumab, Atezolizumab, Atinumab, Atlizumab (tocilizumab), Atorolimumab, Avelumab, Bapineuzumab, Basiliximab, Bavituximab, Begelomab, Belimumab, Benralizumab, Bertilimumab, Besilesomab, Bevacizumab, Bezlotoxumab, Bimagrumab, Bimekizumab, Bivatuzumab mertansine, Bleselumab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Carlumab, Carotuximab, cBR96-doxorubicin immunoconjugate, Cedelizumab, Cergutuzumab amunaleukin, Cetuximab, Ch.14.18, Cixutumumab, Clazakizumab, Clenoliximab, Clivatuzumab tetraxetan, Codrituzumab, Coltuximab ravtansine, Conatumumab, Concizumab, Crenezumab, Crotedumab, CR6261, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Diridavumab, Domagrozumab, Drozitumab, Duligotumab, Dupilumab, Durvalumab, Dusigitumab, Ecromeximab, Eculizumab, Edobacomab, Edrecolomab, Efalizumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Etaracizumab, Etrolizumab, Evinacumab, Evolocumab, Exbivirumab, Fanolesomab, Faralimomab, Farletuzumab, Fasinumab, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalzumab, Ibritumomab tiuxetan, Icrucumab, Idarucizumab, IMAB362, Imalumab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iratumumab, Isatuximab, Itolizumab, Ixekizumab, Keliximab, Labetuzumab, Lampalizumab, Lanadelumab, Landogrozumab, Laprituximab emtansine, Lebrikizumab, Lemalesomab, Lendalizumab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, MABp1, Mapatumumab, Margetuximab, Mavrilimumab, Matuzumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Namilumab, Naratuximab emtansine, Narnatumab, Natalizumab, Navicixizumab, Navivumab, Nebacumab, Necitumumab, Nemolizumab, Nerelimomab, Nesvacumab, Nimotuzumab, Nivolumab, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oregovomab, Orticumab, Otelixizumab, Otlertuzumab, Oxelumab, Ozanezumab, Ozoralizumab, Pagibaximab, Palivizumab, Pamrevlumab, Vectibix, Pankomab, Panobacumab, Parsatuzumab, Pascolizumab, Pasotuxizumab, Pateclizumab, Patritumab, Pembrolizumab, Pemtumomab, Perakizumab, Pertuzumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Sofituzumab vedotin, Solanezumab, Sonepcizumab, Sontuzumab, Stamulumab, Suvizumab, Tabalumab, Tacatuzumab tetraxetan, Talizumab, Tamtuvetmab, Tanezumab, Taplitumomab paptox, Tarextumab, Tefibazumab, Tenatumomab, Teneliximab, Teplizumab, Teprotumumab, Tesidolumab, Tetulomab, Tezepelumab, TGN1412, Ticilimumab (tremelimumab), Tildrakizumab, Tigatuzumab, Timolumab, Tisotumab vedotin, TNX-650, Tocilizumab (atlizumab), Toralizumab, Tosatoxumab, Tositumomab, Tovetumab, Tralokinumab, Trastuzumab, Trastuzumab emtansine, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, Zolimomab aritox, and any combination thereof.

In some embodiments, the antibody or antibody fragment or bi-specific antibody has a molecular weight (MW) of 15-150 kD, 15-50 kD, 100-120 kD, 100-150 kD, 100-200 kD, 100-250 kD, 150-200 kD, 150-250 kD, 200-250 kD. Each possibility represents a separate embodiment. In some embodiments, the antibody has an MW of at least 100 kD, at least 110 kD, at least 120 kD, at least 130 kD, at least 140 kD, at least 150 kD, at least 160 kD, at least 180 kD, at least 200 kD, at least 250 kD. Each possibility represents a separate embodiment. In some embodiments, the antibody has an MW of 150-200 kD. In some embodiments, the antibody has an MW of 130-180 kD. In some embodiments, the antibody has an MW of 140-160 kD.

In some specific embodiments, the antibody has a MW of 150-200 kD and the respective polymeric linker comprises PEG having a MW of at least 1,000 Da, at least 2,000 Da, at least 2,500 Da or at least 3,000 Da. In some embodiments, the antibody has a MW of 150-200 kD and the respective polymeric comprises PEG having a MW of at most 2,000 Da, at most 2,500 Da, at most 3,000 Da, at most 3,500 Da, at most 4,000 Da, at most 5,000 Da or at most 6,000 Da. In some embodiments, the antibody has a MW of 150-200 kD and the respective polymeric linker comprises PEG having a MW of between 1,000 Da to 4,000 Da. In some such embodiments, the internalizing moiety is insulin with a MW of 5-6 kD and the third polymeric linker comprises PEG having a MW of at least 4,000 Da.

In some embodiments, the first and/or the second immunoglobulin is a therapeutic agent that is effective in treating a tumor, cancer or malignancy located outside the brain. In some embodiments, the first and/or the second immunoglobulin is an antibody used for the treatment or diagnosis of cancer, wherein the cancer is other than a brain cancer.

In some embodiments, at least one of the first and the second immunoglobulin is a labeling molecule. The term “labeling molecule”, as used herein, refers to a molecule that is capable of producing a signal detectable by suitable detection means, such as but not limited to radioactive molecules and fluorescent molecules. In some embodiments, the labeling molecule has diagnostic applications. In some embodiments, the labeling molecule is a diagnostic agent. In some embodiments, the labeling molecule is a small molecule. In some embodiments, the labeling molecule is an antibody.

According to the principles of the present invention, the multifunctional system enables the synchronized co-delivery of two immunoglobulins into a specific region, tissue, or cells of the body, in particular into tumor cells or specific spatial area of a tumor, located outside the brain or outside the CNS. In some embodiments, at least one of the first and the second immunoglobulins has a poor membrane penetration in its original, free, form. In some embodiments, both the first and the second immunoglobulins have a poor membrane penetration in their original, free, form.

In some embodiments, each one of the first and the second immunoglobulins is a therapeutic agent having a therapeutic or immune activity against cancer or against a non-CNS related disease or disorder. One of the advantages of co-delivery systems is the possibility to induce synergistic effects. Regarding co-delivery of different active agents, the therapeutic results can either be additive (i.e., the result is that expected by combining the effects of each drug separately) or synergistic (i.e., the combination produces more-significant benefits than that expected by adding the separate effects). In some embodiments, the combination of the first and the immunoglobulins produces an additive therapeutic effect. In other embodiments, the combination of the first and the second immunoglobulins produces a synergistic therapeutic effect.

In some embodiments, the first immunoglobulin is a therapeutic antibody, and the second active agent is a targeting antibody that can bind a specific surface receptor or ligand and can thus target the system to a specific body region or to a particular cell population within the body, leading to enhanced and focused treatment. In some related embodiments, said second antibody further has a therapeutic or immune activity relevant to a malignancy or to a non-CNS related disease or disorder.

In some embodiments, at least one of the first and the second immunoglobulins is a molecule having intracellular targeting capabilities, i.e., a molecule that targets an intracellular macromolecule. In some related embodiment, said immunoglobulin is conjugated to the core particle through a cleavable linker.

It is known that complex diseases, including cancers, are often multifactorial and involve redundant or synergistic action of disease mediators or upregulation of different receptors, including crosstalk between their signaling networks (Kontermann, R. In: MAbs. Taylor & Francis, 2012. p. 182-197). Consequently, blockade of multiple, different pathological factors and pathways may result in a significantly improved therapeutic efficacy. This result can be achieved by combining different immunoglobulins, or use of the dual targeting strategies.

In some embodiments, both the first and the second immunoglobulins can bind a specific surface receptor or ligand, e.g., specific tumor antigen or a specific receptor or other moiety on immune cells. Thus, in some embodiments, the multifunctional system of the invention combines specificities of two different immunoglobulins, e.g., antibodies, in a single system, enabling to simultaneously interfere with different surface receptors or ligands within the tumor or cancer cell. Without wishing to be bound by any theory or mechanism of action, it is hypothesized that dual-targeted particles (e.g., dual-antibody particles) can bring different targets into close proximity, either to support protein complex formation on one cell, or to trigger contacts between cells. In some embodiments, the complex comprises immune cells and cancer cells. In some embodiments, the first and the second active agents are antibodies wherein at least one of said first and second immunoglobulins is a bispecific antibody. Thus, in some embodiments, the multifunctional system of the invention enables to simultaneously interfere with more than two targets. In some related embodiments, at least one of the first and the second immunoglobulins further has a therapeutic activity against a cancer located outside the brain or against a non-CNS related disease or disorder. In some embodiments, both the first and the second active immunoglobulins further have a therapeutic activity against a cancer located outside the brain, or against a non-CNS related disease or disorder.

In some embodiments, each one of the first immunoglobulin and the second immunoglobulin is an antibody or an active fragment thereof comprising at least the antigen binding site, provided that the first and the second antibodies or antibody fragments are different. In some related embodiments, the first active agent and the second active agent comprise different antibodies. In other related embodiments, the first immunoglobulin and the second immunoglobulin comprise or consists of different fragments of the same antibody. For example, in some embodiments, the first immunoglobulin comprises or consists of the Fab region of an antibody and the second comprises or consists of the Fc region of the same antibody. In other embodiments, the first immunoglobulin comprises or consists of a whole antibody (e.g., IgG) and the second immunoglobulin comprises or consists of a fragment of the same antibody. For example, in some embodiments, the first immunoglobulin comprises or consists of a whole antibody (e.g., IgG) and the second immunoglobulin comprises or consists of an Fc region of the same antibody.

According to some embodiments, the core particle is a gold nanoparticle. According to some embodiments, the first linear polymeric linker is a thiolated PEG3500 acid or thiolated PEG35000 amine. According to some embodiments, the second linear polymeric linker is a thiolated PEG3500 acid or thiolated PEG3500 amine. According to some embodiments, the third linear polymeric linker is a thiolated PEG5000 acid or thiolated PEG5000 amine. According to some embodiments, the penetration enhancing moiety is insulin.

According to some embodiments, the core particle is a gold nanoparticle. According to some embodiments, the first linear polymeric linker is a thiolated PEG3500 acid or thiolated PEG35000 amine. According to some embodiments, the second linear polymeric linker is a thiolated PEG1000 acid or thiolated PEG1000 amine. According to some embodiments, the third linear polymeric linker is a thiolated PEG5000 acid or thiolated PEG5000 amine. According to some embodiments, the penetration enhancing moiety is insulin.

In some embodiments, the multifunctional particle further comprises at least one additional immunoglobulin that is attached to the core particle through an additional polymeric linker. The different possibilities of the at least one additional active agent and the respective polymeric linker are similar to those described above for the first and the second active agents, and the first and second polymeric linker.

In some embodiments, the invention provides a plurality of multifunctional particles as described above in all embodiments thereof.

### Preparation Process

According to another aspect, there is provided a process for preparation of the multifunctional particle of the invention, in all embodiments thereof as described above, the process comprising the steps of:
a) partially coating a surface of a core particle with a first polymeric linker followed by conjugating the first polymeric linker to a first immunoglobulin;
b) partially coating the surface of the core particle with a second polymeric linker followed by conjugating the second polymeric linker to a second immunoglobulin; and
c) partially coating the surface of the core particle with a third polymeric linker followed by conjugating the third polymeric linker to a penetration enhancing moiety, wherein steps (a), (b) and (c) can be performed in any order.

The term “partially coating”, as used herein, refers to conjugating a plurality of the respective polymeric linkers to the surface of a particle, such that the plurality of linkers partly covers the surface of the particle at a density level below the saturation level of the naked particle.

Any method known in the art can be used for determining the amount of polymer required for achieving full-density (i.e., 100%) coating of a particle, and accordingly the amount needed for partial coating. For example, adding different amounts of polymer to the particle solution and measuring the concentration of the free polymer in supernatants after centrifugation is a widely used method. Alternatively, any characterization method that is sensitive to changes in coating density can be used, such as zeta potential and DLS. Furthermore, theoretical calculations can be performed to determine the amount of polymer needed to achieve complete coating according to the surface area of the particle. For example, it was previously shown that a thiol-PEG molecule occupies a footprint area 0.35 nm² on gold nanoparticle surface (Qian, Ximei, et al. Nature biotechnology 26.1 (2008): 83-90). Accordingly, the amount of a thiol-PEG linker required to cover 100% of the surface of a gold nanoparticle (GNP) can be calculated based on the mean diameter of the GNP.

In some embodiments, each one of the first polymeric linker, the second polymeric linker and the third polymeric linker is added in an amount suitable for covering between 5-70%, 5-60%, 5-40%, 8-60%, 10-60%, 10-55%, 10-50%, 10-40%, 10-30%, 10-25%, 10-20%, 15-60%, 15-55%, 15-50%, 15-45%, 15-40%, 15-30%, 15-25%, 15-20%, 2-10%, 2-20%, 2-50%, 2-60%, 2-70%, 5-10%, 5-20%, 5-70%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention.

In some embodiments, step (a) comprises coating between 5-70%, 5-60%, 5-40%, 8-60%, 10-60%, 10-55%, 10-50%, 10-40%, 10-30%, 10-25%, 10-20%, 15-60%, 15-55%, 15-50%, 15-45%, 15-40%, 15-30%, 15-25%, 15-20%, 2-10%, 2-20%, 2-50%, 2-60%, 2-70%, 5-10%, 5-20%, 5-70%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention.

In some embodiments, step (b) comprises coating between 5-70%, 5-60%, 5-40%, 8-60%, 10-60%, 10-55%, 10-50%, 10-40%, 10-30%, 10-25%, 10-20%, 15-60%, 15-55%, 15-50%, 15-45%, 15-40%, 15-30%, 15-25%, 15-20%, 2-10%, 2-20%, 2-50%, 2-60%, 2-70%, 5-10%, 5-20%, 5-70%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention.

In some embodiments, step (c) comprises coating between 5-70%, 5-60%, 5-40%, 8-60%, 10-60%, 10-55%, 10-50%, 10-40%, 10-30%, 10-25%, 10-20%, 15-60%, 15-55%, 15-50%, 15-45%, 15-40%, 15-30%, 15-25%, 15-20%, 2-10%, 2-20%, 2-50%, 2-60%, 2-70%, 5-10%, 5-20%, 5-70%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention.

In some embodiments, steps (a)-(c) are performed sequentially, in any order. A person skilled in the art would be able to determine the optimal order of the steps according to different parameters, e.g., the core particle type, the specific polymeric linkers, the immunoglobulins used, the penetration enhancing moiety and the like. In some embodiments, the process further comprises centrifugation after each one of steps (a), (b) and (c).

In some embodiments, the first polymeric linker and the second polymeric linker are identical. In some related embodiments, steps (a) and (b) are performed simultaneously, by partially coating the surface of the core particle with the first and second polymeric linkers together, and then conjugating the first and the second active agents to the polymeric linkers. In some related embodiments, the step of partially coating the surface of the core particle with the first and second polymeric linkers together, comprises coating between 10-70%, 10-60%, 10-40%, 10-60%, 10-60%, 10-55%, 10-50%, 10-45%, 10-40%, 10-30%, 10-25%, 10-20%, 15-60%, 15-55%, 15-50%, 15-45%, 15-40%, 15-30%, 15-25%, 15-20%, 10-20%, 10-50%, 10-70%, 20-50%, 20-40%, 30-50%, 30-60%, 30-70%, 50-60% or 50-70% of the surface of the core particle. Each possibility represents a separate embodiment of the present invention. In further related embodiments, conjugating the first and the second active agents to the polymeric linkers comprises adding a mixture of the first and the second active agents in the desired molar ratio, to the particle solution.

In some embodiments, the process further comprises partially coating the surface of the core particle with a fourth polymeric linker. In some embodiments, the fourth polymeric linker is a monofunctional linker used to cap access reactive groups on the particle.

According to related embodiments, there is provided a process for preparation of a multifunctional particle, the process comprising the steps of:
a) partially coating a surface of a core particle with a first polymeric linker followed by conjugating the first polymeric linker to a first immunoglobulin;
b) partially coating the surface of the core particle with a second polymeric linker followed by conjugating the second polymeric linker to a second immunoglobulin;
c) partially coating the surface of the core particle with a third polymeric linker followed by conjugating the third polymeric linker to the penetration enhancing moiety; and
d) partially coating the surface of the core particle with a fourth polymeric linker, wherein the fourth polymeric linker is a monofunctional linker, wherein steps (a), (b), (c) and (d) can be performed in any order.

According to some embodiments, the particle is a gold nanoparticle (GNP) and the process for the preparation of multifunctional gold nanoparticles, comprises the sequential steps of: (a) reduction of HAuCl₄ to produce gold nanoparticles; (b) simultaneous incubation of the reduced GNPs with one monofunctional linker and three heterofunctional linkers, to bind them to the gold nanoparticles; (c) activation of the acid groups of the linkers; (d) conjugation of the penetration enhancing moiety; and (e) conjugation of two different antibodies by incubating with a solution comprising their mixture.

According to some embodiments, analysis of the GNPs is performed following each step using methods known in the art.

According to some embodiments, the monofunctional linker is mPEG-SH. According to a specific embodiment, the monofunctional linker is mPEG5000-SH or mPEG6000-SH and it is added to cover about 80% of particle surface.

According to some embodiments, the heterofunctional likers are COOH-PEG-SH. According to some embodiments, one heterofunctional liker is COOH-PEG5000-SH and it is added in a concentration to cover about 15% of particle surface. According to some embodiments, the other heterofunctional liker is COOH-PEG3500-SH and it is added in a concentration to cover about 5% of particle surface.

According to some embodiments, activation of the GNPs coated with the different linkers, is performed by mixing the GNPs with (1-ethyl-3-(3-dimethylaminopropyl) carbodiimide HCl (EDC).

The core particle, the first polymeric linker, the second polymeric linker, the third polymeric linker, the fourth polymeric linker, the penetration enhancing moiety, and the first and second immunoglobulins suitable for use in the preparation process are those described hereinabove in connection with the various aspects and embodiments of the co-delivery system.

### Pharmaceutical Compositions

In yet another aspect, there is provided a pharmaceutical composition comprising the multifunctional particle according to the various embodiments described hereinabove and a pharmaceutically acceptable carrier. In some embodiments, the pharmaceutical composition comprises a plurality of multifunctional particles according to the various embodiments described hereinabove, and a pharmaceutically acceptable carrier.

As used herein, a “pharmaceutically acceptable formulation,” “pharmaceutical composition” or “pharmaceutically acceptable composition” may include any of a number of carriers such as solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Pharmaceutical compositions containing the presently described particles as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

A composition may comprise different types of carriers depending on whether it is to be administered in solid, liquid or aerosol form, and whether it needs be sterile for such routes of administration as injection. A person of ordinary skill in the art would be familiar with techniques for generating sterile solutions for injection or application by any other route. Sterile injectable solutions are prepared by incorporating the active compounds in the required amount in an appropriate solvent with various other ingredients familiar to a person of skill in the art.

The carrier may comprise, in total, from about 0.1% to about 99.99999% by weight of the pharmaceutical compositions presented herein.

According to some embodiments, the pharmaceutical composition is formulated for systemic administration. According to some embodiments, the pharmaceutical composition is formulated for systemic administration selected from intravenous and intranasal administration. According to some embodiments, the pharmaceutical composition is formulated for intravenous administration. According to some embodiments, the pharmaceutical composition is formulated for intranasal administration.

The compositions contemplated herein may take the form of solutions, suspensions, emulsions, aerosols, combinations thereof, or any other pharmaceutical acceptable composition as would commonly be known in the art.

In some embodiments, the carrier is a solvent. For a non-limiting example, the composition may be disposed in the solvent. Such a solvent includes any suitable solvent known in the art such as water, saline, phosphate-buffered saline.

The formulation of the composition may vary depending upon the route of administration. For parenteral administration in an aqueous solution, for example, the solution should be suitably buffered if necessary and the liquid diluent first rendered isotonic with sufficient saline or glucose. Sterile aqueous media which can be employed will be known to those of skill in the art in light of the present disclosure.

Supplementary active ingredients can also be incorporated into the compositions. For human administration, preparations should meet sterility and general safety and purity standards as required by FDA Office of Biologics standards. Administration may be by any known route.

In certain embodiments, a pharmaceutical composition includes at least about 0.001 g to about 1 g of the particle disclosed herein per kilogram of a subject. In certain embodiments, a pharmaceutical composition includes at least about 0.001 g to about 0.5 g of the particle disclosed herein per kilogram of a subject.

The pharmaceutical composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. The composition must be stable under the conditions of manufacture and storage, and preserved against the contaminating action of microorganisms, such as bacteria and fungi. It will be appreciated that exotoxin contamination should be kept minimally at a safe level, for example, less than 0.5 ng/mg protein.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. In many cases, it will be preferable to include isotonic agents, such as, for example, sugars, sodium chloride or combinations thereof.

In other embodiments, a nasal solutions or sprays, aerosols or inhalants may be used. Nasal solutions are usually aqueous solutions designed to be administered to the nasal passages in drops or sprays.

Solid compositions for oral administration are also contemplated. In these embodiments, the solid composition may comprise, for example, solutions, suspensions, emulsions, tablets, pills, capsules, sustained release formulations, buccal compositions, troches, elixirs, suspensions, syrups, or combinations thereof.

Sterile injectable solutions are prepared by incorporating the active compounds (e.g., nanoparticles) in the required amount in the appropriate solvent with various other ingredients enumerated above. The liquid medium should be suitably buffered if necessary and the liquid diluent first rendered isotonic prior to injection with sufficient saline or glucose.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art.

According to some embodiments, a pharmaceutical composition, comprising at least one delivery system according to the present invention, and a pharmaceutical composition, comprising an additional immuno-modulator or a kinase inhibitor, are used in treatment of cancer by separate administration.

Toxicity and therapeutic efficacy of the compositions described herein can be determined by standard pharmaceutical procedures in cell cultures or experimental animals, e.g., by determining the IC50 (the concentration which provides 50% inhibition) and the maximal tolerated dose for a subject compound. The data obtained from these cell culture assays, and animal studies can be used in formulating a range of dosages for use in humans. The dosage may vary depending inter alia upon the dosage form employed, the dosing regimen chosen, the composition of the agents used for the treatment and the route of administration utilized, among other relevant factors. The exact formulation, route of administration and dosage can be chosen by the individual physician in view of the patient's condition. Depending on the severity and responsiveness of the condition to be treated, dosing can also be a single administration of a slow-release composition, with course of treatment lasting from several days to several weeks or until cure is affected or diminution of the disease state is achieved. The amount of a composition to be administered will, of course, be dependent on the subject being treated, the severity of the affliction, the manner of administration, the judgment of the prescribing physician, and all other relevant factors.

### Therapeutic and Diagnostic Use of the Composition

According to some aspects, there is provided a pharmaceutical composition comprising the multifunctional particle of the invention, for use in the co-delivery of a first and a second immunoglobulins to a specific population of cell or to a location within a body or tissue, but outside of a brain, and in some embodiments, outside of the CNS, of a subject in need thereof.

According to some embodiments, the co-delivery is to a primary tumor, malignancy, or to killer cells.

According to other aspects, the present invention provides a method for a synchronized delivery of a first and a second immunoglobulins to a diseased or malignant tissue or cells, outside the CNS, of a subject in need thereof, the method comprises administering to the subject a pharmaceutical composition comprising the multifunctional particle described above in all embodiments thereof.

According to some embodiments, the pharmaceutical composition is for use in treating a cancer, a tumor or cancer cells located outside the brain and in some embodiments outside the CNS.

According to some embodiments, that tumor is a primary tumor.

According to other embodiments, the cancer cells are metastases located outside the brain. According to yet other embodiments, the cancer cells are metastases located outside the CNS.

The term “cancer” refers to or describes the physiological condition in mammals that is typically characterized by unregulated cell growth. Examples of cancer include but are not limited to, carcinoma, adenocarcinoma, blastoma, and sarcoma. More particular examples of such cancers include breast, colon, prostate, melanoma, lung, thyroid, hepatic, bladder, renal, cervical, pancreatic, ovarian, uterus, sarcoma, biliary, or endometrial cancer.

According to some aspects and embodiments, there is provided a method for treating cancer in a subject in need thereof, the method comprising administering the pharmaceutical composition of the invention to the subject.

According to some aspects and embodiments, there is provided a method for monitoring a cancer in a subject in need thereof, the method comprising administering the pharmaceutical composition of the invention to the subject and imaging a specific region or tissue of the subject, or a specific population of cells. In some related embodiments, the pharmaceutical composition comprises the multifunctional particle described above wherein the core particle is a gold nanoparticle and the imaging is performed using CT to enable detection of the multifunctional particle within a specific body region, other than the CNS. In other embodiments, the pharmaceutical composition comprises the multifunctional particle described above wherein at least one immunoglobulin is a labeling molecule, e.g., labelled with a fluorescent or radioactive molecule which allows detection by a suitable imaging modality. In some embodiments, the method for monitoring the disease or disorder comprises repeated administrations and/or repeated imaging sessions.

According to some embodiments, the method of treating cancer is part of a treatment regimen comprising at least one additional anticancer treatment. According to certain embodiments, the additional anticancer therapy is selected from surgery, chemotherapy, radiotherapy, or immunotherapy.

Pharmaceutical compositions according to the present invention may be used as part of combined therapy with at least one anti-cancer agent.

As used herein the term “combination”, “combined therapy” or “combination treatment” can refer either to concurrent administration of the articles to be combined or sequential administration of the articles to be combined. As described herein, when the combination refers to sequential administration of the articles, the articles can be administered in any temporal order.

According to some embodiments, the additional anti-cancer agent is an immuno-modulator, an activated lymphocyte cell, a kinase inhibitor or a chemotherapeutic agent.

According to some embodiments, the anti-cancer agent is selected from the group consisting of an antimetabolite, a mitotic inhibitor, a taxane, a topoisomerase inhibitor, a topoisomerase II inhibitor, an asparaginase, an alkylating agent, an antitumor antibiotic, and combinations thereof.

According to some embodiments, the anti-cancer agent is an immuno-modulator, whether agonist or antagonist, such as antibody against an immune checkpoint molecule.

Checkpoint immunotherapy blockade has proven to be an exciting new venue of cancer treatment. Immune checkpoint pathways consist of a range of co-stimulatory and inhibitory molecules which work in concert in order to maintain self-tolerance and protect tissues from damage by the immune system under physiological conditions. Tumors take advantage of certain checkpoint pathways in order to evade the immune system. Therefore, the inhibition of such pathways has emerged as a promising anti-cancer treatment strategy. According to some embodiments, the immuno-modulator is selected from the group consisting of: an antibody inhibiting CTLA-4, an anti-human programmed cell death protein 1 (PD-1), PD-L1 and PD-L2 antibody, an activated cytotoxic lymphocyte cell, a lymphocyte activating agent, an antibody against CEACAM, an antibody against TIGIT, and a RAF/MEK pathway inhibitor, an anti lymphocyte activation gene 3 (LAG3) antibody, an anti CD137 antibody, an anti OX40 (CD134) antibody, and an antibody against killer cell immunoglobulin-like receptors (KIR).

In some embodiments, the pharmaceutical composition according to the present invention is for use in enhancing the immune response, namely for increasing the responsiveness of the immune system and inducing or prolonging its memory.

As used herein, the term “subject” refers to any animal (e.g., a mammal), including, but not limited to, humans, non-human primates, rodents, and the like (e.g., which is to be the recipient of a particular treatment). Typically, the terms “subject” and “patient” are used interchangeably, unless indicated otherwise herein.

In some embodiments, the subject is a human subject. In some embodiments, the subject is at risk of being afflicted with cancer. In some embodiments, the subject is diagnosed with cancer. In some embodiments, the subject is diagnosed with a primary cancer located outside the brain. In some embodiments, the subject is diagnosed with a non-CNS-related cancer.

As used herein, a subject at risk of being afflicted with a disease, a disorder, or a medical condition, is a subject that presents one or more signs or symptoms indicative of a disease, a disorder, or a medical condition or is being screened for a disease, a disorder, or a medical condition (e.g., during a routine physical). A subject at risk of being afflicted with a disease, a disorder, or a medical condition, may also have one or more risk factors. A subject at risk of being afflicted with a disease, a disorder, or a medical condition encompasses an individual that has not been previously tested for the disease, disorder, or medical condition. However, a subject at risk of being afflicted with a disease, a disorder, or a medical condition, also encompasses an individual who has received a preliminary diagnosis but for whom a confirmatory test (e.g., biopsy and/or histology) has not been done or for whom the stage of the disease, disorder, or medical condition is not known. The term further includes people who once had the disease, disorder, or medical condition (e.g., an individual in remission).

As used herein, a subject diagnosed with cancer, may be diagnosed using any suitable method, including but not limited to, biopsy, x-ray, blood test, and the diagnostic methods of the present invention.

As used herein, the terms “treatment”, “treating”, or “ameliorating” of a disease, disorder, or condition, refer to alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. The terms as used herein refer to both therapeutic treatment and prophylactic or preventative measures.

Those in need of treatment include those already with the disorder as well as those in which the disorder is to be prevented.

The term “administering” or “administration of” a substance, a compound, an agent, or a pharmaceutical composition to a subject can be carried out using one of a variety of methods known to those skilled in the art. For example, a pharmaceutical composition can be administered enterally or parenterally. Enterally refers to administration via the gastrointestinal tract including per os, sublingually or rectally. Parenteral administration includes administration intravenously, intradermally, intramuscularly, intraperitoneally, subcutaneously, ocularly, sublingually, intranasally, by inhalation, intraspinally, intracerebrally, and transdermally (by absorption, e.g., through a skin duct). An agent or a delivery system can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a pharmaceutical composition. For example, as used herein, a physician who instructs a patient to self-administer a pharmaceutical composition, or to have the composition administered by another and/or who provides a patient with a prescription for a drug is administering the composition to the patient.

Administering the composition to the subject can be done by using any method known to those of ordinary skill in the art. The mode of administering may vary based on the application. For example, the mode of administration may vary depending on the particular cell, body region, or subject to be imaged. For example, administering the composition may be done intravenously, intracerebrally, intracranially, intrathecally, intracerebroventricular, into the substantia nigra or the region of the substantia nigra, intradermally, intraarterially, intraperitoneally, intralesionally, intratracheally, intranasally, intramuscularly, intraperitoneally, subcutaneously, orally, topically, locally, by inhalation (e.g., aerosol inhalation), by injection, by infusion, by subarachnoid infusion, by transmucosal infusion, by intracarotid infusion, by continuous infusion, by localized perfusion bathing target cells directly, via a catheter, via a lavage, or by other method or any combination of the forgoing as would be known to one of ordinary skill in the art.

In some embodiments, the pharmaceutical composition is administered to the subject by a systemic administration route. In some embodiments, the systemic administration is selected from an intravenous (IV) administration and an intranasal (IN) administration. In some embodiments, the particle is administered intravenously. In some embodiments, the particle is administered intranasally.

An effective amount of the pharmaceutical composition is determined based on the intended goal and the subject to be treated. The amount to be administered may also vary based on the particular route of administration to be used. The composition is preferably administered in a safe and effective amount. As used herein, the term “safe and effective amount” refers to the quantity of a composition which is sufficient for the intended goal without undue adverse side effects (such as toxicity, irritation, or allergic response).

In some embodiments, the core particle is a radiosensitizer and the method of treatment the cancer further comprises a step of directing an ionizing irradiation to the tumor cells (in which the particles accumulate) thereby obtaining locally enhanced radiation therapy within the tumor cells.

In some embodiments, the composition is used for thermal ablation of tumor cells in which the composition accumulates using Infra-Red waves, without causing damage to surrounding normal tissues or substantial toxicity to the subject. As used herein, “ablation” refers to the destruction of cells. Methods for irradiating a tissue comprising metal particles for enhancing effects of radiation therapy, are known in the art.

The present invention further discloses methods for diagnosing and prognosing cancer.

According to an aspect, the present invention provides a diagnostic and/or prognostic method of cancer in a subject, the method comprises the step of determining the expression level of a protein in a biological sample of said subject using at least one diagnostic composition as described herein.

The term “biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived there from and the progeny thereof. Additionally, the term may encompass circulating tumor or other cells. The term specifically encompasses a clinical sample, and further includes cells in cell culture, cell supernatants, cell lysates, serum, plasma, urine, amniotic fluid, biological fluids including aqueous humour and vitreous for eyes samples, and tissue samples. The term also encompasses samples that have been manipulated in any way after procurement, such as treatment with reagents, solubilization, or enrichment for certain components. The method of the invention can further comprise the step of comparing said level of expression to a control level.

In some embodiments, the method further comprises a step of imaging a specific region of the subject. In some embodiments, the imaging is performed using an imaging system selected from the group consisting of: computed tomography imaging (CT), X-ray imaging, magnetic resonance imaging (MRI), positron emission tomography (PET), single-photon emission computed tomography (SPECT), ultrasound (US), and any combination thereof.

In some embodiments, the imaging is performed to evaluate accumulation of the co-delivery system in the tissue or tumor of said subject.

In some embodiments, the subject is afflicted with a tumor or a cancer, and the imaging is performed for determining the stage of the disease or disorder. In some embodiments, the subject afflicted with a tumor or cancer was treated with a drug and the imaging method is used for follow-up of the treatment.

In some embodiments, the imaging step is performed 0.5 to 96 hours post the administering step. In some embodiments, the imaging step is performed 0.5 to 48 hours post the administering step. In some embodiments, the imaging step is performed 0.5 to 24 hours post the administering step. In some embodiments, the imaging step is performed 0.5 to 12 hours post the administering step. In some embodiments, the imaging step is performed 1 to 12 hours post the administering step. In some embodiments, the imaging step is performed 1 to 6 hours post the administering step. In some embodiments, the imaging step is performed within 96 hours from the administering step. In some embodiments, the imaging step is performed within 48 hours from the administering step. In some embodiments, the imaging step is performed within 24 hours from the administering step. In some embodiments, the imaging step is performed within 12 hours from the administering step. In some embodiments, the imaging step is performed within 6 hours from the administering step.

### Kits

In some embodiments, the invention provides kits comprising one or more compositions disclosed herein. In some embodiments, the invention provides kits useful for methods disclosed herein. For example, a kit may include a container having a sterile reservoir that houses any composition disclosed herein. In some embodiments, the kit further includes instructions. For example, a kit may include the instructions for administering the composition to a subject (e.g., indication, dosage, methods etc.). In yet another example the kit may include instructions regarding application of the compositions and methods of the invention to imaging systems e.g., computed tomography (CT), ultrasound (US), magnetic resonance imaging (MRI).

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or length, are to be understood to include any integer within the recited range, unless otherwise indicated.

As used herein, the term “about” when combined with a value refers to plus and minus 10% of the reference value. For example, a molecular weight of about 1000 Da refers to a molecular weight of 1000 Da+−100 Da.

It is noted that as used herein and in the appended claims, the singular forms “a,” “an,” and “the” include plural referents unless the context clearly dictates otherwise. Thus, for example, reference to “a particle” includes a plurality of such particles and reference to “the particle” includes reference to one or more particles. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

The term “plurality” means “two or more”, unless expressly specified otherwise.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### EXAMPLES

Generally, the nomenclature used herein, and the laboratory procedures utilized in the present invention include chemical synthesis, molecular, biochemical, microbiological and recombinant DNA techniques. Such techniques are well known in the art, provided throughout this document and/or thoroughly explained in the literature.

### Example 1: Multifunctional Gold Nanoparticles (GNPs) Co-Deliver Two Antibodies

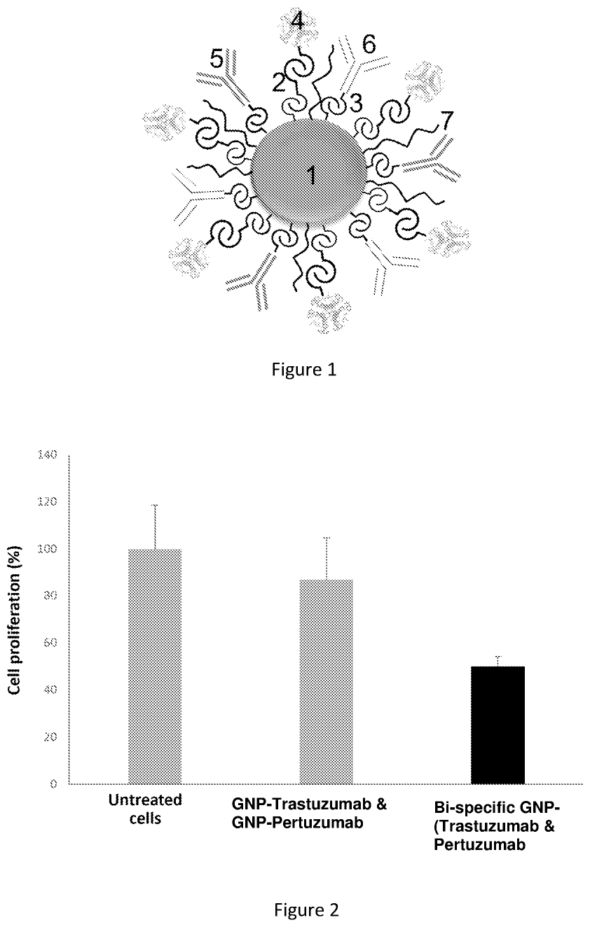

FIG. 1 schematically illustrates a non-limiting exemplary multifunctional particle, showing a gold nanoparticle (GNP; 1) bound to: (i) a first polymeric linker (2) which is conjugated to a penetration enhancing moiety (e.g., insulin) (4); (ii) a second polymeric linker (3) which is conjugated to a first antibody (5) and to a second antibody (6); and (iii) a capping polymer moiety (7).

### Preparation and Characterization of GNPs Conjugated to Anti-IgG1, Anti-Iba1 and Insulin (IgG1 & Iba1 &Ins-GNPs)

GNP synthesis: 20 nm spherical GNPs were prepared by citrate reduction of HAuCl₄. A total of 414 μl of 50% w/v of HAuCl₄ solution in 200 ml distilled water was boiled in an oil bath on a heating plate while being stirred. After boiling, 4.04 ml of a 10% sodium citrate solution were added and the mixture was stirred while boiling for another 10 minutes. The solution was removed from the plate and after cooling to room temperature, the solution was centrifuged until precipitation of the nanoparticles.

Conjugation of PEG5000 to the GNPs: GNPs were first partially coated (60% of particle surface) with mPEG-SH (˜5 kDa; 40% of particle surface) and a heterofunctional HS-PEG-COOH (˜5 kDa; 20% of particle surface). The amount of mPEG-SH and HS-PEG-COOH required for the partial coating was derived from theoretical calculations based on the finding that thiol-PEG molecule occupies a footprint area 0.35 nm² on gold nanoparticle surface (Qian, Ximei, et al. Nature biotechnology 26.1 (2008): 83-90). Conjugation was performed by adding a mixture of HS-PEG-COOH (193 μl, 50 mg/ml) and mPEG-SH (387 μl, 50 mg/ml) to the GNP solution and mixing for two hours. The solution was then ultra-centrifuged at 15,000 RPM for 20 minutes and then again at 20,000 RPM for 15 minutes. The precipitate, containing the PEG-coated GNPs (total 60% coating) was transferred to a vial.

Conjugation of insulin: In order to facilitate the transport of the multifunctional GNP through membranes, insulin was covalently conjugated to the carboxylic group of the HS-PEG-COOH by addition of excess amount of insulin on ice together with EDC (1-ethyl-3-(3-dimethylaminopropyl) carbodiimide HCl) and NHS (N-hydroxysulfosuccinimide sodium salt) followed by mixing for two hours. Then, the solution was centrifuged twice at 15,000 RPM for 30 minutes (maintained at a cool temperature) and the lower phase, containing the Ins-PEG-GNPs was transferred into a vial.

Conjugation of PEG3500 to the GNPs: In order to further conjugate antibodies to the GNPs, 271 μl of HS-PEG-COOH (˜3.5 kDa) solution (50 mg/ml) were added to the partially coated GNPs to coat the remaining 40% of particle surface. The solution was then mixed for two hours at 4° C. followed by repetitive centrifugation at 15,000 RPM for 30 minutes.

Conjugation of anti-IgG1 and anti-Iba1: Fluorescently labeled anti-Iba1 and Fluorescently labeled anti-IgG1 were covalently conjugated to free carboxylic groups of the HS-PEG-COOH (˜3.5 kDa) by addition of 1:1 molar mixture of fluorescently labeled anti-Iba1 (Ab195032-Rb Mono & Hu IBA-1-647) and fluorescently labeled anti-IgG1 (mouse Monoclonal IgG1 Alexa Fluor 488 Isotype Control Clone 11711) together with EDC and NHS. The solution was then stirred for 2 hours at 4° C. followed by centrifugation to remove unbound antibodies, until a final concentration of 25 mg/ml Au was reached.

To confirm the chemical conjugation of the antibodies, the multifunctional dual antibody nanoparticles were imaged using a fluorescent microscope.

For control experiments, similar particles were prepared with only one conjugated antibody, i.e., anti-IgG1 or anti-Iba1. Percentage coverage of the different coating molecules in the control particles: 20% PEG5000 (conjugated to insulin), 20% PEG3500 (conjugated to the respective antibody) and 60% mPEG5000.

### Example 2: Preparation of Multifunctional GNPs Coated with Insulin and with Two Anti HER2 Antibodies, Trastuzumab and Pertuzumab

As a non-limiting example, gold nanoparticles carrying two different anti HER2 antibodies and insulin, were produced. The exemplary particles were coated with a polymeric layer (2-3 in FIG. 1) comprising two polymeric linkers (5-S-PEG-C(O)—, ˜5 kDa and —S-PEGC (O)—, ˜3.5 kDa) the first linker was conjugated to insulin (4) and the second linker is conjugated to two different anti HER2 antibodies (5-6) Trastuzumab and Pertuzumab. An additional (7) polymeric moiety (—S-PEG-O—CH3, ˜6 kDa) is used to populate the surface or the gold nanoparticle to control the density of the other moieties on the particle.

### GNP Synthesis

20 nm spherical GNPs were prepared by citrate reduction of HAuCl₄. A total of 414 ul of 42.77% w/v of HAuCl₄ in 200 ml double distilled water (DDW) was boiled in an oil bath on a heating plate while being stirred. After boiling, 4.04 ml of a 10% w/v trisodium citrate in DDW were added. The solution was removed from the oil bath and left to cool while being stirred at room temperature.

Conjugation of COOH-PEG5000-SH, COOH-PEG3500-SH and mPEG6000-SH to the GNPs.

GNPs were incubated with mPEG5000-SH (˜5 kDa; 80% of particle surface), a heterofunctional COOH-PEG5000-SH (˜5 kDa; 15% of particle surface) and a heterofunctional COOH-PEG3500-SH (˜3.5 kDa; 5% of particle surface). The amount of PEG moieties that are required for proportional coating was derived from theoretical calculations based on the findings that thiol-PEG molecule occupies a surface area of 0.35 nm² on gold nanoparticle surface (Qian, Ximei, et al. Nature biotechnology 26.1 (2008): 83-90). Conjugation was performed by adding a mixture of COOH-PEG5000-SH (127 μl, 50 mg/ml in DDW), mPEG5000-SH (809 μl, 50 mg/ml in DDW) and COOH-PEG3500-SH (30 μl, 50 mg/ml in DDW) to the GNP solution and mixing overnight. The solution was then centrifuged at 50,000G for 20 minutes, then the precipitants were redispersed in DDW and centrifuged at 50,000G for 20 minutes. The precipitate, containing the PEG-coated GNPs was transferred to a vial.

Activation of the GNPs was performed by mixing the GNPs with EDC (1-ethyl-3-(3-dimethylaminopropyl) carbodiimide HCl, 30 mg/ml in DDW, 100 μl) and sulfo-NHS (N-hydroxysulfosuccinimide sodium salt, 30 mg/ml in DDW, 100 μl) followed by centrifugation at 50,000G for 20 minutes. The precipitate, containing the activated COOH groups was transferred to a vial.

Conjugation of insulin to HS-PEG5000-COOH was then performed by addition of insulin (195IU, 100IU/ml) to the GNPs solution for 3 h. Then a solution containing trastuzumab and pertuzumab (total 15 mg) was inserted into 2 ml borate buffer (PH 8, 0.1M) and later added to the GNP-insulin solution for the conjugation of the remaining COOH-PEG3500-SH while mixing over-night. The solution was then centrifuged at 10,000G, 20 min. Followed by redissolving the precipitants in saline and then centrifuged at 10,000G, 20 min.

The GNPs coated with antibodies and insulin (Abs&Ins-GNPs) were characterized following each step of preparation using Dynamic Light Scattering (DLS). The hydrodynamic size and Zeta potential of the GNPs confirmed successful coating.

The quantification of the antibodies (Abs) and insulin attached to the PEG groups on the GNPs was tested by enzyme-linked immunosorbent assay (ELISA) tested on the supernatants containing the unbound proteins left from centrifugation.

### Example 3: The In Vitro Effect of Dual-Antibody GNPs on HER2 Positive Cancer Cells

GNPs conjugated to insulin and the different anti-HER2 antibodies Trastuzumab and Pertuzumab were prepared according to the protocol described in Examples 3. For comparison, GNPs conjugated to insulin and a single antibody (either Trastuzumab or Pertuzumab) were also prepared.

BT474, a HER2 positive breast cancer cell line was used to determine the effect of GNPs conjugated to each of the antibodies as a single therapy and their combined effect. Cells were treated with Trastuzumab-GNPs, Pertuzumab-GNPs, or Trastuzumab & Pertuzumab-GNPs at different concentration. Untreated cells served as control.

The effect of the various treatments on the cells was examined using cell cycle arrest determination, apoptosis and proliferation assays. Each treatment was performed in triplicates.

In a specific experiment, the cells were incubated for 5 days with the following conditions: 1. Control-untreated, 2. Mixture of GNPs with Trastuzumab & GNPs with Pertuzumab, (each of the GNPs was prepared with insulin as the penetration enhancing moiety) 3. Bi-specific GNPs with both Trastuzumab & Pertuzumab conjugated to the same particle (and with insulin). Cell proliferation assay was conducted to find out the efficacy of the complex in inhibiting the growth of the tumor cells. As demonstrated in FIG. 2, the bi-specific GNP complex showed a better efficacy in inhibiting cancer cell proliferation, than the mixture of the two antibodies each conjugated to a separate particle.

### Example 4: The In Vitro Effect of Dual-Antibody GNPs on Breast Cancer Cells

The bi-specific nanocomplex was tested with human breast cancer cell lines BT474 and MCF7 that have high (BT474) and relatively low (MCF7) expression of HER2 receptors.

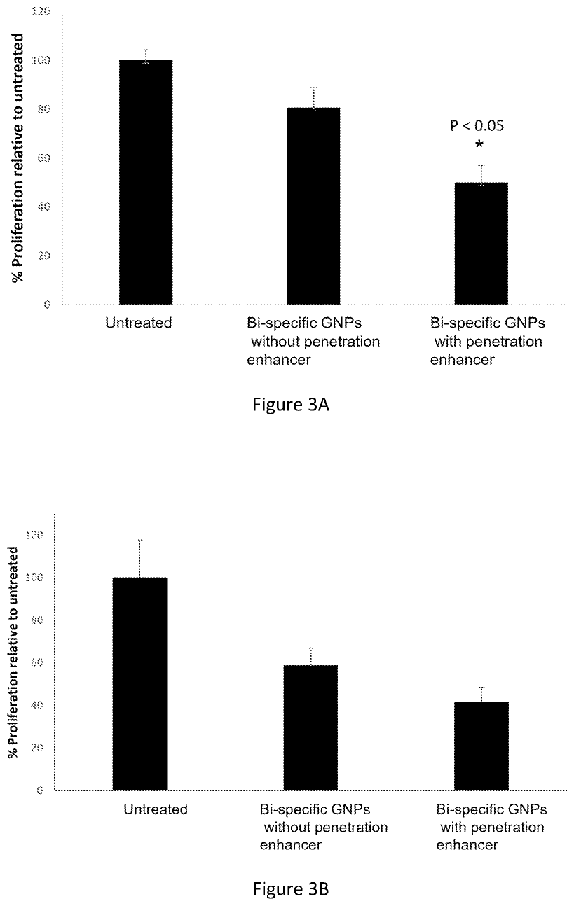

The cells were incubated for 5 days with the following conditions: (i) Control-untreated; (ii) Bi-specific GNPs with both Trastuzumab & Pertuzumab anti HER2 antibodies conjugated to the same particle without a transporter or penetration enhancing molecule; and (iii) Bi-specific GNPs with both Trastuzumab & Pertuzumab conjugated to the same particle in addition to insulin molecules. Cell proliferation assay was conducted to determine the efficacy of the complex in inhibiting the growth of the tumor cells. As shown in FIGS. 3A and 3B, the bi-specific GNP complex that included the insulin molecules on the delivery system were more effective in inhibiting the proliferation of the cells, while GNPs with the antibodies but without the insulin had less effect. Without wishing to be bound to any mechanism of action, it is proposed that the insulin molecules on the delivery system bind to insulin receptor on cancer cells and enhance contact and binding of the anti HER2 antibodies to cells expressing HER2 receptor, which are infrequent on these cells, thereby inhibiting proliferation of these cancer cells.

### Example 5: Determining the Optimal Density of Antibodies Per Particle

Different compositions of the nanocomplex with increasing number of attached antibodies were tested in vitro, for inhibition of proliferation of BT474 human breast cancer cells that overexpress HER2 receptors.

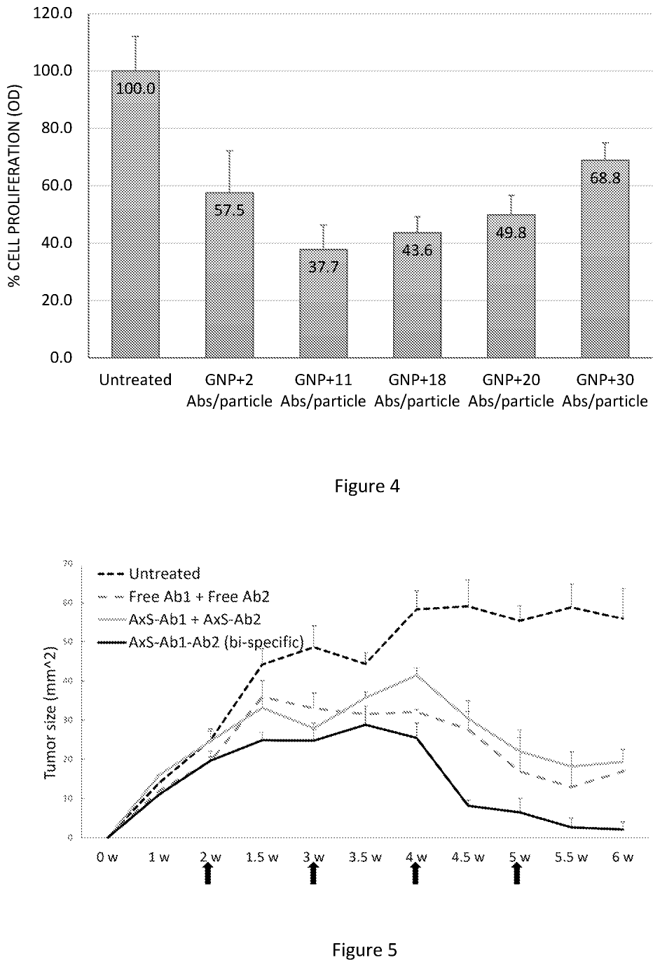

The cells were incubated for 5 days with the following conditions: (i) Control-untreated; (ii) GNPs with 2 antibodies attached to each particle; (iii) GNPs with 11 antibodies attached to each particle; (iv) GNPs with 18 antibodies attached to each particle; (v) GNPs with 20 antibodies attached to each particle and (vi) GNPs with 30 antibodies attached to each particle. Cell proliferation assay was conducted to determine the efficacy of the different complexes in inhibiting the growth of the HER2 positive tumor cells. As depicted in FIG. 4, complexes carrying 10 to 20 antibodies per particle where most active in inhibiting cancer cell proliferation with 62-50% inhibition.

### Example 6: Bispecific GNP Complex for Cancer Treatment: An In Vivo Study

The efficacy of the platform as a multi-functional drug carrier was tested in vivo for inhibition of tumor growth. For this experiment the antibodies Trastuzumab and Pertuzumab, considered the first line combination treatment for breast cancer tumors were used. The GNPs were conjugated with both antibodies and insulin that enhances tumor targeting and penetration through binding to insulin receptors present on tumor cells and tested in a subcutaneous metastatic breast cancer tumor mice model, using BT474 cells. Two weeks post tumor inoculation the mice were divided into 4 groups; a control group, a group that received the mixture of the free antibodies, a group that received a mixture of GNPs one with Trastuzumab and others with Pertuzumab, and the fourth group received bi-functional GNPs (with both antibodies conjugated to the same particle). The treatment material was injected IP once a week, for 4 consecutive weeks. The results demonstrated in FIG. 5, indicate that the multi-functional particles are significantly (p<0.01) more efficient than both the free antibodies and the mixture of the monofunctional particles in reducing the tumor size.

### Example 7: Bispecific GNP Immunotherapeutic Complex for Inhibition of Tumor Cell Proliferation

In this experiment, the immunotherapeutic antibodies: anti PD-1, targeted to receptors on T-cells, and anti PD-L1, targeted to receptors on the tumor cells, were tested when conjugated alone or together with GNPs.

The first step included activation of the immune cells, and then 6 days later, the immune cells were incubated with H1299 lung cancer cells to test the combined activity in inhibition of growth of the tumor cells.

Anti-CD3 and anti-CD28 were added to peripheral blood mononuclear cells (PBMCs) to trigger T-cells activation. Then, the following substances were added to the cell wells (each one in a triplicate) and the incubation duration was 6 days: (i) mixture of free anti PD-1 and anti PD-L1 antibodies; (ii) GNPs conjugated with both anti PD-1 and anti PD-L1 antibodies produced according to the method of example 3.

After 6 days, medium was removed to detect cytokines and PBMCs were collected and added to H1299 lung cancer cells to determine T-cell cytotoxicity on the target cells. The PBMCs were incubated with the H1299 cells for 18 hours, and then cell proliferation was measured by ELISA.

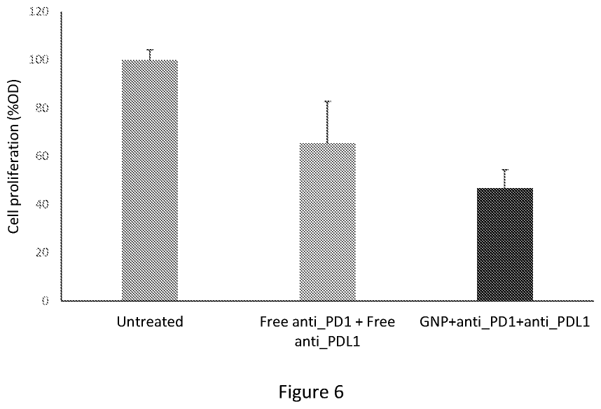

As demonstrated in FIG. 6, the mixture of PBMC cells and cancer cells in the presence of the multi-specific GNPs carrying anti PD-1 and anti PD-L1 had a better efficacy in inhibition of cancer cell's proliferation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-50. (canceled)

51. A method for preventing, treating and/or monitoring a cancer comprising a primary tumor or tumor metastases located outside the brain of a subject in need thereof, the method comprising administering to the subject a pharmaceutical composition comprising a multifunctional particle comprising:
(a) an inorganic particle bound to at least: (i) a first linear hetrofunctional polymeric linker; (ii) a second linear hetrofunctional polymeric linker; (iii) a third linear hetrofunctional polymeric linker; and (iv) a fourth polymeric monofunctional linker;
(b) a first immunoglobulin molecule covalently conjugated to the first linear polymeric linker;
(c) a second immunoglobulin molecule covalently conjugated to the second linear polymeric linker; and
(d) a penetration enhancing moiety conjugated to the third linear polymeric linker;
wherein the first and the second immunoglobulin molecules are distinct; and wherein about 2 to 400 immunoglobulin molecules in total, are conjugated to each particle, through the first and second linkers.

52. The method of claim 51, wherein 2 to 20 immunoglobulin molecules in total, are conjugated to each particle, through the first and second linkers.

53. The method of claim 51, wherein 2 to 10 immunoglobulin molecules in total, are conjugated to each particle, through the first and second linkers.

54. The method of claim 51, wherein the primary tumor is a solid tumor or wherein the solid tumor is selected from a breast, lung, melanoma, prostate, bladder, pancreatic, ovarian tumor, resistant tumor and a metastatic tumor

55. The method of claim 51, wherein the primary tumor is a breast tumor selected from the group consisting of: a tumor expressing HER2, a HER2-low tumor, and a triple negative breast tumor.

56. The method of claim 51, wherein the first, the second, the third and the fourth polymeric linkers are non-cleavable under physiological conditions.

57. The method of claim 51, wherein the molecular weight of each of the first and the second linear polymeric linkers is 3500 Da or more.

58. The method of claim 51, wherein the molecular weight of each of the third and fourth polymeric linkers is within a range of 4500-8000 Da.

59. The method of claim 51, wherein the first and the second linear polymeric linkers are bound to the inorganic particle through a semi-covalent bond, and the first and the second immunoglobulin molecules are conjugated to the respective linear polymeric linkers through an amide bond.

60. The method of claim 51, wherein the fourth linker is conjugated to about 90% to 99% of a total surface capacity of the inorganic particle.

61. The method of claim 51, wherein at least one of the first and second immunoglobulin molecules is capable of binding a cancer-specific or cancer-associated cell-surface antigen on a tumor cell, or

62. The method of claim 51, wherein the first and the second immunoglobulin molecules are each independently an antibody or antibody fragment capable of binding a cancer-specific or cancer-associated cell-surface antigen on a tumor cell.

63. The method of claim 51, wherein the first and the second immunoglobulin molecules are each independently an antibody or antibody fragment, and wherein the first and the second antibodies or antibody fragments are capable of binding HER2.

64. The method according to claim 51, further comprising administering or performing at least one additional anti-cancer therapy selected from chemotherapy, immunotherapy, biotherapy, hormonal therapy, radiation therapy, bone-marrow transplantation, surgery, and any combination thereof.

65. The method according to claim 51, wherein treatment results in preventing or reducing formation, growth or spread of metastases in the subject.

66. A method for simultaneous delivery of at least two immunoglobulin molecules to a specific region, cells, or tissue outside of a brain of a subject, the method comprising
administering to the subject a pharmaceutical composition comprising a multifunctional particle, wherein the multifunctional particle comprises:
(a) an inorganic particle bound to at least: (i) a first linear polymeric linker; (ii) a second linear polymeric linker; (iii) a third linear polymeric linker; and (iv) a fourth polymeric monofunctional linker;
(b) a first immunoglobulin molecule conjugated to the first linear polymeric linker;
(c) a second immunoglobulin molecule, covalently conjugated to the second linear polymeric linker; and
(d) a penetration enhancing moiety conjugated to the third linear polymeric linker;
wherein the first and the second immunoglobulin molecules are distinct.

67. A method for preventing, treating and/or monitoring a primary tumor or tumor metastases located outside the brain of a subject in need thereof, the method comprising administering to the subject a pharmaceutical composition comprising a multifunctional particle, wherein the multifunctional particle comprises:
(a) an inorganic particle bound to at least: (i) a first linear polymeric linker; (ii) a second linear polymeric linker; (iii) a third linear polymeric linker; and (iv) a fourth polymeric monofunctional linker;
(b) a first immunoglobulin molecule conjugated to the first linear polymeric linker;
(c) a second immunoglobulin molecule, covalently conjugated to the second linear polymeric linker; and
(d) a penetration enhancing moiety conjugated to the third linear polymeric linker;
wherein the first and the second immunoglobulin molecules are distinct.
